(19) United States
(12) Patent Application Publication
(10) Pub. No.: US 2016/0374944 A1
(43) Pub. Date: Dec. 29, 2016

(54) COMPOSITIONS AND METHODS FOR TREATING AND PREVENTING CANCER BY TARGETING AND INHIBITING CANCER STEM CELLS

(71) Applicant: Rakesh Srivastava, Overland Park, CA (US)

(72) Inventor: Rakesh Srivastava, Overland Park, CA (US)

(21) Appl. No.: 15/163,551

(22) Filed: May 24, 2016

Related U.S. Application Data
(63) Continuation-in-part of application No. 13/476,840, filed on May 21, 2012, now abandoned.
(60) Provisional application No. 61/488,001, filed on May 19, 2011.

(51) Int. Cl. A61K 9/16; A61K 31/105; A61K 31/352; A61K 31/7068; A61K 31/26; A61K 31/05

(52) U.S. Cl. CPC A61K 9/1611; A61K 31/26; A61K 31/05; A61K 31/352; A61K 31/7068; A61K 31/105

(57) ABSTRACT

The invention includes compositions and methods for treating cancer comprising administering to a subject in need a pharmaceutically effective dose of a cancer stem cell inhibitor, methods of inhibiting the growth of cancer stem cells or tumor initiating cell comprising administering to a subject in need a pharmaceutically effective amount of a pharmaceutical composition for inhibiting cancer stem cells. The pharmaceutical composition includes various combinations of sulforaphane, resveratrol, mangostine, honokiol, diallyltrisulphide and gemcitabine.

### CROSS REFERENCE TO RELATED APPLICATIONS

This application is a continuation in part (CIP) application of the U.S. patent application Ser. No. 13/476,840, which claims the benefit of U.S. Provisional Appl. No. 61/488,001, filed May 19, 2011, the content of which is incorporated by reference herein in its entirety.

### FIELD OF THE INVENTION

The present invention relates to compositions and methods for inhibiting cancer stem cells, and resulting treatments for cancer.

### BACKGROUND OF THE INVENTION

Cancer stem cells (CSCs), progenitor cells, and tumor initiating cells give rise to tumor bulk through continuous processes of self-renewal and differentiation. CSCs are highly tumorigenic, have a tendency to self-renew, and express certain cell surface markers; for example, pancreatic CSCs express CD133/CD44/CD24/ESA. See also Table 1. CSCs are also a cause of tumor relapse, drug resistance, and chemo- and radio-therapy failure. Strategies are being developed towards the targeted destruction of CSCs while sparing the physiological stem cells, which may lead to marked improvement in patient outcome. By altering the expression of genes and pathways by novel agents and approaches, various cancers can be prevented and treated by targeting CSCs and progenitor cells. Selective and targeted elimination of the CSCs may be a key for cancer therapy and prevention.

Cancer of the pancreas is the fourth leading cause of cancer death in the United States. Approximately 32,000 Americans die each year from cancer of the pancreas. With an overall 5-year survival rate of 3%, pancreatic cancer has one of the poorest prognoses among all cancers. Aside from its silent nature and tendency for late discovery, pancreatic cancer also shows unusual resistance to chemotherapy and radiation. CSCs have recently been proposed to be the cause of cancer chemotherapy failure, as well as the cause of initiation and progression. Only 20% of pancreatic cancer patients are eligible for surgical resection, which currently remains the only potentially curative therapy. The operations are very complex, and unless performed by surgeons specially trained and experienced in this procedure, they can be associated with very high rates of operative morbidity and mortality. Unfortunately, many cancers of the pancreas are not resectable at the time of diagnosis. There are limited treatment options available for this disease because chemo- and radio-therapies are largely ineffective, and metastatic disease frequently redevelops even after surgery.

TABLE 1
Specific cell surface markers for human CSCs

| No. | Type of cancer | Cell surface markers |
|---|---|---|
| 1. | Pancreatic | CD133+, CD44+, |
| | | CD24+, Lgr5 |
| 2. | Prostatic | CD44+, integrin |
| 3. | Breast | CD44+, CD24⁻/^{low} |
| 4. | Ovarian | CD44+, MyD88+ |
| 5. | Colon | CD133+, CD44+, |
| | | CD166+, |
| | | E-CAM^{high}, Lgr5 |
| 6. | AML | CD34+, CD38− |
| 7. | Myeloproliferative | CD117+ |
| | disorder | |
| 10 | Glioblastoma | CD133+, Nestin, |
| | | CD15+ |
| 11 | Medulloblastoma | CD133+ |
| 12 | Hepatocellular cancer | CD133+ |
| 13 | Head and neck | CD44+ |
| | squamous cell | |
| | carcinoma | |
| 14 | Metastatic melanoma | CD20+ |
| 15 | Bone sarcomas | Stro-1+, CD105+, |
| | | CD44+ |
| 16 | Lung | CD133+ |

Currently, there is no effective drug for the treatment of pancreatic cancer. Gemcitabine, a common drug used in the treatment of pancreatic cancer, is effective in only 30% pancreatic cancer patients with survival less than 5 years. Furthermore, the toxicity of new drugs, which are in the clinical trials, is very high. Therefore, effective and non-toxic drugs are urgently needed for the prevention and treatment of pancreatic cancer.

### SUMMARY OF THE INVENTION

The present invention generally relates to compositions and methods for treating various cancers including, but not limited to, breast, prostrate, brain, lung, mesothelioma, melanoma, multiple myeloma, colon, kidney, ovarian, and pancreatic cancer, leukemia, and lymphoma. More particularly, the present invention generally relates to methods of treating cancer using cancer stem cell inhibitors.

In one aspect, the present invention provides a method of treating cancer comprising administering to a subject in need a pharmaceutically effective dose of a stem cell inhibitor.

In another aspect, the present invention provides a method of inhibiting the growth of cancer stem cells comprising administering to a subject in need a pharmaceutically effective dose of a stem cell inhibitor.

In another aspect, the present invention provides a method of enhancing the biological effects of chemotherapeutic drugs on cancer cells comprising administering to a subject in need thereof, along with a pharmaceutically effective dose of a chemotherapeutic drug or a chemopreventive agent, a pharmaceutically effective dose of a cancer stem cell inhibitor.

In some embodiments, the cancer stem cell inhibitor may be one or more of rottlerin, embelin, ellagic acid, sulforaphane, resveratrol, honokiol, curcumin, diallyltrisulfide, benzyl isothiocyanate, quercetin, epigallocatechin gallate (EGCG), SAHA, m-Carboxycinnamic acid bis-hydroxamine, MS-275, SAHA/vornostat, m-Carboycinnamic acid bis-hydroxamine, 5-aza-2′-deoxycytidine, benzyl selenocyanate (BSC), benzyl isothiocyanate (BITC), phenyl isothiocyanate (PITC), anthothecol, sanguinarine, and mangostine, or a pharmaceutically acceptable salt or ester thereof.

In some embodiments, the cancer stem cells are from cancers including breast cancer, prostrate cancer, brain cancer, lung cancer, mesothelioma, melanoma, multiple myeloma, colon cancer, kidney cancer, head and neck cancer, ovarian cancer, pancreatic cancer, leukemia, and lymphoma.

In some embodiments, the cancer stem cell inhibitor also kills cancer cells.

The features and advantages of the present invention will be apparent to those skilled in the art. While numerous changes may be made by those skilled in the art, such changes are within the spirit of the invention.

### BRIEF DESCRIPTION OF THE DRAWINGS

Some specific example embodiments of the invention may be understood by referring, in part, to the following description and the accompanying drawings.

FIG. 1 is the molecular structure of gemcitabine.

FIG. 2 is a drawing depicting the molecular structure of rottlerin.

FIG. 3 is a drawing depicting the molecular structure of embelin.

FIG. 4 is a drawing depicting the molecular structure of ellagic acid.

FIG. 5 is a drawing depicting the molecular structure of sulforaphane.

FIG. 6 shows graphs showing the results of cell viability studies. In particular, the effect of rottlerin on the growth of human pancreatic cancer cells, and cancer stem cells, is shown. Pancreatic cancer cells (AsPC-1, PANC-1 and MIA PaCa-2) and pancreatic cancer stem cells (CSCs) were treated with rottlerin for 3 days, and cell viability was measured by XTT assay. Data represent mean±SD.

FIG. 7 shows graphs showing the results of cell viability studies. In particular, the effect of embelin on the growth of human pancreatic cancer cells is shown. Pancreatic cancer cells (AsPC-1, PANC-1 and MIA PaCa-2) were treated with embelin for 3 days, and cell viability was measured by XTT assay. Data represent mean±SD.

FIG. 8 shows graphs showing the results of cell viability studies. In particular, the effect of ellagic acid on the growth of human pancreatic cancer cells is shown. Pancreatic cancer cells (AsPC-1 and MIA PaCa-2) were treated with ellagic acid for 3 days, and cell viability was measured by XTT assay. Data represent mean±SD.

FIG. 9 shows graphs showing the effect of embelin on prostate CSCs. In particular, embelin is shown to inhibit spheroid and colony formation, and induce caspase-3 and apoptosis. (A) Prostate CSCs were grown in CSC medium and treated with embelin (0-6 μM) for 7 days to obtain primary spheroids. At the end of incubation period, spheroids were collected, reseeded and treated with embelin for another week to obtain secondary spheroids. Cell viability in spheroids was measured by trypan blue assay at the end of 7 and 14 days. Data represent mean±SD. *, %, &, # and **=significantly different from controls, P<0.05. (B) Prostate CSCs were seeded in soft agar and treated with embelin (0-6 μM) for 21 days. At the end of incubation period, numbers of colonies were counted. *, % and &=significantly different from control, P<0.05. (C and D) Activation of caspase-3 and induction of apoptosis is shown. Prostate CSCs were treated with embelin (0-6 μM), and caspase-3 activity at 24 h and apoptosis at 48 h were measured; as described *, #, % or &=significantly different from control, P<0.05.

FIG. 10 is a graph showing the effect of embelin on prostate CSCs. In particular, embelin is shown to inhibit the expression of Bcl-2, survivin and XIAP in prostate CSCs. Prostate CSCs were treated with embelin (0-6 μM) for 24 h, and the expression of Bcl-2, survivin and XIAP was measured by the q-RT-PCR. Data represent mean±SD. *=significantly different from respective controls, P<0.05. Data were normalized with GAPDH.

FIG. 11 is a graph showing the regulation by embelin of Nanog and Oct3/4 in prostate CSCs. Prostate CSCs were treated with embelin (0-6 μM) for 24 h. The expression of Nanog and Oct3/4 was measured by qRT-PCR. Data represent mean±SD. *=significantly different from control, P<0.05. Data were normalized with GAPDH.

FIG. 12 shows graphs showing the regulation by embelin of the Shh pathway in prostate CSCs. (A-C) Prostate CSCs were treated with embelin (0-6 μM) for 24 h. The expression of Gli1, Gli2, Patched-1, Patched-2 and SMO was measured by qRT-PCR. Data represent mean±SD. *, # and &=significantly different from control, P<0.05. Data were normalized with GAPDH. (D) Gli transcriptional activity. Prostate CSCs were transduced with Gli-responsive GFP/firefly luciferase viral particles (pGreen Fire1-Gli with EF1, System Biosciences). After transduction, culture medium was replaced and CSCs were treated with embelin (0-6 μM) for 24 h. Gli-responsive reporter activity was measured using a luciferase assay (Promega Corporation). Data represent mean±SD. *, # and &=significantly different from control, P<0.05.

FIG. 13 shows graphs showing the inhibition of invasion, migration and EMT markers by embelin. (A and B) Prostate CSCs were treated with embelin (0-6 μM) for 24 h. Invasion and migration of CSCs were measured as we described. *, # and &=significantly different from respective controls, P<0.05. (C) Expression of Snail and N-cadherin was measured by the qRT-PCR. Data represent the mean±S.D. *=significantly different from respective controls, P<0.05.

FIG. 14 shows graphs showing that rottlerin inhibits spheroid and colony formation, and induces caspase-3 and apoptosis. (A) Prostate CSCs were grown in CSC medium and treated with rottlerin (0-2 μM) for 7 days to obtain primary spheroids. At the end of incubation period, spheroids were collected, reseeded and treated with rottlerin for another week to obtain secondary spheroids. Cell viability in spheroids was measured by trypan blue assay at the end of 7 and 14 days. Data represent mean±SD. *, % and &=significantly different from controls, P<0.05. (B) Prostate CSCs were seeded in soft agar and treated with rottlerin (0-2 μM) for 21 days. At the end of incubation period, numbers of colonies were counted. *, % and &=significantly different from control, P<0.05. (C and D) Activation of caspase-3 and induction of apoptosis. Prostate CSCs were treated with rottlerin (0-2 μM) for 24 h, and caspase-3 activity and apoptosis were measured as we described. *, #, %, and &=significantly different from control, P<0.05.

FIG. 15 is a graph showing that rottlerin inhibits the expression of survivin, XIAP, Bcl-2 and Bcl-X_{L} in prostate CSCs. Prostate CSCs were treated with rottlerin (0-2 μM) for 24 h, and the expression of survivin, XIAP, Bcl-2 and Bcl-X_{L} was measured by the q-RT-PCR. Data represent mean±SD. *=significantly different from respective controls, P<0.05. Data were normalized with GAPDH.

FIG. 16 is a graph showing the regulation by rottlerin of cMyc, Nanog, Oct3/4 and Sox-2 in prostate CSCs. Prostate CSCs were treated with rottlerin (0-1 μM) for 24 h. The expression of cMyc, Nanog, Oct3/4 and Sox-2 was measured by qRT-PCR. Data represent mean±SD. * and #=significantly different from control, P<0.05. Data were normalized with GAPDH.

FIG. 17 graphs showing the regulation of Shh, Notch and TGFβ pathways by rottlerin. FIG. 17F shows the results of immunofluorescence analysis of Gli1 and Gli2 expression in prostate CSCs (A-C) Prostate CSCs were treated with rottlerin (0-2 μM) for 24 h. The expression of Patched-1, Patched-2, SMO, Gli1 and Gli2 was measured by qRT-PCR. Data represent mean±SD. *, #, and &=significantly different from control, P<0.05. Data were normalized with GAPDH. (D) Prostate CSCs were treated with rottlerin (0-1 μM) for 24 h. The expression of Notch1, Notch3 and JAG1 was measured by qRT-PCR. (E) TCF/LEF-1, Gli and Notch reporter activities. Prostate CSCs were transduced with a mixture of TCF/LEF1-, Gli-, or Notch-responsive firefly luciferase construct and Renilla luciferase construct (40:1) along with lipofectamine. After transduction, medium was changed and CSCs were treated with rottlerin (0-2 μM) for 24 h. Reporter activity was measured using a dual luciferase assay (Promega Corporation). *=significantly different from control, P<0.05. (F) Immunofluorescence analysis of Gli1 and Gli2 expression in prostate CSCs. Green=Gli1 or Gli2; red=nucleus.

FIG. 18 shows photographs and a graph showing that rottlerin inhibits cell viability in spheroids and colony formation by pancreatic CSCs. (A) Pancreatic CSCs were grown in six-well ultralow attachment plates (Corning Inc., Corning, N.Y.) at a density of 1,000 cells/ml in DMEM supplemented with 1% N2 Supplement (Invitrogen), 2% B27 Supplement (Invitrogen), 20 ng/ml human platelet growth factor (Sigma-Aldrich), 100 ng/ml EGF (Invitrogen) and 1% antibiotic-antimycotic (Invitrogen) at 37° C. in a humidified atmosphere of 95% air and 5% CO₂ and treated with rottlerin (0-2 μM) for 7 days to obtain primary spheroids. At the end of incubation period, spheroids were collected, reseeded and treated with rottlerin for another week to obtain secondary spheroids. (B) Cell viability in spheroids was measured by trypan blue assay at the end of 7 and 14 days from the above experiment. Data represent mean±SD. *, #, and **=significantly different from controls, P<0.05.

FIG. 19 is a graph showing the regulation by rottlerin of cMyc, Nanog, Oct-4, and Sox-2 in pancreatic CSCs. Pancreatic CSCs were treated with rottlerin (0-1 μM) for 24 h. The expression of Nanog, Sox-2 and cMyc was measured by qRT-PCR and normalized to GAPDH. Data represent mean±SD. **=significantly different from control, P<0.05.

FIG. 20 shows graphs showing the regulation by rottlerin of the Shh pathway in pancreatic CSCs. (A) Pancreatic CSCs were treated with rottlerin (0-1 μM) for 24 h. The expression of Patched-1, Smo and Gli-2 was measured by qRT-PCR. Data represent mean±SD. **, and **=significantly different from control, P<0.05. (B) Pancreatic CSCs were transduced with Gli-responsive GFP/firefly luciferase viral particles (pGreen Fire1-Gli with EF1, System Biosciences). After transduction, culture medium was replaced and CSCs were treated with rottlerin (0-2 μM) for 24 h. Gli-responsive reporter activity was measured using a luciferase assay (Promega Corporation). Data represent mean±SD. *, @ and $=significantly different from control, P<0.05.

FIG. 21 shows graphs showing that rottlerin induces apoptosis, activates caspase-3/-7, and inhibits the expression of Bcl-2, XIAP and Survivin in pancreatic CSCs. (A and B) Induction of apoptosis and activation of caspase-3/-7. Pancreatic CSCs were treated with rottlerin (0-2 μM) for 48 h, and apoptosis and caspase-3/-7 activity were measured by XTT and colorometric assay, respectively. Data represent mean±SD. *, # and @=significantly different from control, P<0.05. (C) Pancreatic CSCs were treated with rottlerin (0-1 μM) for 48 h, and the expression of Bcl-2, XIAP and Survivin was measured by qRT-PCR and normalized to GAPDH. Data represent mean±SD. **, and ##=significantly different from control, P<0.05.

FIG. 22 shows graphs, and FIG. 22C are the results of an in vitro Matrigel invasion assay, showing the regulation of EMT markers by rottlerin in pancreatic CSCs. (A and B) Pancreatic CSCs were treated with rottlerin (0-1 μM) for 24 h. The expression of Zeb-1 and Slug was measured by the qRT-PCR and normalized to GAPDH. Data represent the mean±S.D. *=significantly different from respective controls, P<0.05. (C) In vitro Matrigel invasion assay. CSCs were plated onto the Matrigel-coated membrane in the top chamber of the transwell and treated with rottlerin (0-2 μM) for 48 h. Cells invaded to the lower chamber were fixed with methanol, stained with crystal violet and photo micrographed.

FIG. 23 shows graphs showing that resveratrol, curcumin, honokiol, and diallyl trisulphide inhibit cell viability in brain cancer stem cells. Brain CSCs were treated with resveratrol (0-20 μM), curcumin (0-20 μM), honokiol (0-20 μM) and diallyl trisulphide (0-10 μM) for 3 days, and cell viability was measured by staining with trypan blue using Vi-CELL analyzer (Beckman Counter).

FIG. 24 shows graphs showing that sulforaphane, rottlerin, EGCG, and embelin inhibit cell viability in brain cancer stem cells. Brain CSCs were treated with sulforaphane (0-20 μM), rottlerin (0-1 μM), EGCG (0-40 μM) and embelin (0-5 μM) for 48 h, and cell viability was measured by staining with trypan blue using Vi-CELL analyzer (Beckman Counter).

FIG. 25 shows graphs showing that resveratrol, curcumin, honokiol, and diallyl trisulphide inhibit cell viability in prostate cancer stem cells. Prostate CSCs were treated with resveratrol (0-20 μM), curcumin (0-20 μM), honokiol (0-20 μM) and diallyl trisulphide (0-10 μM) for 3 days, and cell viability was measured by staining with trypan blue using Vi-CELL analyzer (Beckman Counter).

FIG. 26 shows graphs showing that sulforaphane, rottlerin, EGCG, and embelin inhibit cell viability in prostate cancer stem cells. Prostate CSCs were treated with sulforaphane (0-20 μM), rottlerin (0-5 μM), EGCG (0-40 μM) and embelin (0-1 μM) for 3 days, and cell viability was measured by staining with trypan blue using Vi-CELL analyzer (Beckman Counter).

FIG. 27 shows graphs showing that resveratrol, curcumin, honokiol, and diallyl trisulphide inhibit cell viability in pancreatic cancer stem cells. Pancreatic CSCs were treated with resveratrol (0-20 μM), curcumin (0-20 μM), honokiol (0-20 μM) and diallyl trisulphide (0-20 μM) for 3 days, and cell viability was measured by staining with trypan blue using Vi-CELL analyzer (Beckman Counter).

FIG. 28 shows graphs showing that sulforaphane, rottlerin, EGCG, and embelin inhibit cell viability in pancreatic cancer stem cells. Pancreatic CSCs were treated with sulforaphane (0-20 μM), rottlerin (0-2 μM), EGCG (0-60 μM) and embelin (0-5 μM) for 3 days, and cell viability was measured by staining with trypan blue using Vi-CELL analyzer (Beckman Counter).

FIG. 29 shows graphs showing that sulforaphane, diallyl trisulphide, resveratrol, and curcumin inhibit cell viability in breast cancer stem cells. Breast CSCs were seeded in 96-well plate and treated with sulforaphane, diallyl trisulphide, resveratrol and curcumin for 3 days. At the end of incubation period, CSCs were harvested and cell viability was measured by XTT assay.

FIG. 30 shows graphs showing that rottlerin, EGCG, embelin, and honokiol inhibit cell viability in breast cancer stem cells. Breast CSCs were seeded in 96-well plate and treated with rottlerin, EGCG, embelin, and honokiol for 3 days. At the end of incubation period, breast CSCs were harvested and cell viability was measured by XTT assay.

FIG. 31 shows graphs showing that chromatin modulators inhibit cell viability and promote apoptosis in pancreatic cancer stem cells. (A) Pancreatic CSCs were treated with SAHA (3 and 5 μM) and 5-Aza-dc (2 and 4 μM) and cell viability was measured at 48 h by staining with trypan blue using Vi-CELL analyzer (Beckman Counter). (B) Pancreatic CSCs were untreated (a) or treated with SAHA (b) or 5-Aza-dC (c) for 48 h, and apoptosis was measured by staining with annexin-PI using Accuri Flow Cytometer. (C) Caspase-3/7 activity was measured in pancreatic CSCs treated with SAHA (0.5 and 2 μM) or 5-Aza-dC (1 and 3 μM) for 25 h. Data represent mean±SD. * and $=significantly different from control, P<0.05.

FIG. 32 shows graphs showing that sulforaphane, rottlerin, and embelin inhibit tumor growth in NOD/SCID/IL2R gamma mice. (A) Pancreatic CSCs were orthotopically implanted in pancreas of NSG mice, and treated with or without sulforaphane 20 mg/kg, for 6 weeks, (B and C) Pancreatic CSCs were xenografted sub-cutaneously in NSG mice, and treated with or without rottlerin and embelin 20 mg/kg, for 6 weeks, (D and E) Prostate CSCs were xenografted sub-cutaneously in NSG mice, and treated with or without rottlerin and embelin 20 mg/kg, for 6 weeks. At the end of the treatment, weights of tumors in treated mice were compared with control mice.

FIG. 33 shows graphs showing that benzyl selenocyanate (BSC), honokiol and phenyl isothiocyanate (PITC) inhibit cell viability in cancer stem cells. (A-B) Pancreatic CSCs were treated with BSC (0-20 μM) and honokiol (0-10 μM); (C-D) Prostate CSCs were treated with PITC (0-20 μM) and BSC (0-20 μM) for 3 days. Cell viability was measured by XTT assay.

FIG. 34 shows graphs showing that PITC, BSC, sulforaphane and honokiol inhibit cell viability in breast cancer stem cells. (A-D) Breast CSCs were treated with PITC (0-20 μM), BSC, (0-20 μM), sulforaphane (0-20 μM) and honokiol (0-10 μM) for 3 days, and cell viability was measured by XTT assay.

FIG. 35 is a graph showing that rottlerin inhibits cell viability in breast cancer stem cells. Breast CSCs were treated with rottlerin (0-1 μM) for 3 days, and cell viability was measured by XTT assay.

FIG. 36 shows graphs showing that sulforaphane and honokiol inhibit cell viability in brain cancer stem cells. (A-B) Brain CSCs were treated with sulforaphane (0-20 μM), and honokiol (0-20 μM) for 3 days, and cell viability was measured by XTT assay.

FIG. 37 shows graphs and photographs showing the effects of EGCG on tumor spheroids and cell viability of pancreatic cancer stem cells (CSCs). (A) Pancreatic CSCs were seeded in suspension and treated with EGCG (0-60 μM) for 7 days. Pictures of spheroids formed in suspension were taken by a microscope. (B) Pancreatic CSCs were seeded in suspension and treated with EGCG (0-60 μM) for 7 days. At the end of incubation period, spheroids were collected, and dissociated with Accutase (Innovative Cell Technologies, Inc.). For secondary spheroids, cells were reseeded and treated with EGCG (0-60 μM) for 7 days. Cell viability was measured by trypan blue assay. Data represent mean±SD. *, &, @, or #=significantly different from respective controls, P<0.05. (C) EGCG inhibits colony formation by CSCs. Pancreatic CSCs were seeded in soft agar and treated with various doses of EGCG and incubated at 4° C. for 21 days. At the end of incubation period, colonies were counted. Data represent mean±SD. *, & or #=significantly different from respective controls, P<0.05.

FIG. 38 shows graphs showing the regulation of caspase-3/7 activity, apoptosis and apoptosis-related proteins by EGCG on CSCs derived from human primary pancreatic tumors. (A) Regulation of caspase-3/7 activity by EGCG. CSCs were treated with EGCG (0-60 μM) for 24 h, and caspase-3/7 activity was measured as per manufacturer's instructions. Data represent mean±SD. *, # or %=significantly different from control, P<0.05. (B) Regulation of apoptosis by EGCG. CSCs were treated with EGCG (0-60 μM) for 48 h, and apoptosis was measured by TUNEL assay. Data represent mean±SD. *, # or %=significantly different from control, P<0.05. (C) Regulation of apoptosis-related proteins. Pancreatic CSCs were treated with EGCG (0-60 μM) for 36 h. Real time PCR (q-RT-PCR) was performed to examine the expression of Bcl-2, survivin, XIAP, and GAPDH. Data represent mean±SD. *, # or %=significantly different from control, P<0.05.

FIG. 39 shows graphs showing the regulation of pluripotency maintaining transcription factors by EGCG in pancreatic cancer stem cells. (A) Pancreatic CSCs were treated with EGCG (0-60 μM) for 36 h. At the end of incubation period, cells were harvested and the expression of Nanog, Sox-2, c-Myc and Oct-4 was measured by the q-RT-PCR. Data represent mean±SD. *, #, or %=significantly different from respective controls, P<0.05. (B) Nanog shRNA enhances the inhibitory effects of EGCG on CSC's spheroid viability. Pancreatic CSCs were transduced with either scrambled shRNA or Nanog shRNA expressing lentiviral vector (pLKO.1), and cell lysates were collected and western blot analysis was performed using anti-Nanog antibody (data not shown). CSC/scrambled and CSC/Nanog shRNA were seeded as described above and treated with EGCG (0-60 μM). After 7 days, spheroids were collected and cell suspensions were prepared and viable cells were counted by trypan blue assay. Data represent mean±SD. *, &, @, #, ** or %=significantly different from control, P<0.05.

FIG. 40A and FIG. 40C are graphs and photographs showing the inhibition of components of sonic hedgehog pathway, Gli transcription and nuclear translocation by EGCG. (A) Inhibition of components of sonic hedgehog pathway and Gli transcription. Pancreatic CSCs were treated with EGCG (0-60 μM) for 36 h. The expression of Smothened (Smo), patched 1 (PTCH1), patched 2 (PTCH2), was measured by q-RT-PCR. Data represent mean±SD. *, #, or %=significantly different from respective controls, P<0.05. (B) Inhibition of Gli1 and Gli2 expression and Gli transcription. Pancreatic CSCs were treated with EGCG (0-60 μM) for 36 h. The expression of Gli1 and Gli2 was measured by q-RT-PCR. Gli reporter activity. CSCs were transduced with Gli-responsive GFP/firefly luciferase viral particles (pGreen Fire1-Gli with EF1, System Biosciences). After transduction, culture medium was replaced and CSCs were treated with EGCG (0-60 μM) for 24 h. Gli-responsive reporter activity was measured by luciferase assay (Promega Corporation). Data represent mean±SD. *, #, % or &=significantly different from respective controls, P<0.05. (C) EGCG inhibits nuclear translocation of Gli1 and Gli2. Pancreatic CSCs were treated with or without EGCG (40 or 60 μM) for 24 h. At the end of incubation period, CSCs were fixed with paraformaldehyde, permeabilized with titron X100, and blocked with 5% normal goat serum. Cells were then treated with either anti-Gli1 or anti-Gli2 antibody, followed by secondary antibody plus DAPI. Stained cells were mounted and visualized under a fluorescence microscope. Blue fluorescence of DAPI was changed to red color for a better contrast.

FIG. 41 shows graphs showing the regulation of epithelial mesenchymal transition factors, migration, invasion and TCF/LEF activity by EGCG in pancreatic CSCs. (A) Pancreatic CSCs were treated with EGCG (0-60 μM) for 48 h. At the end of incubation period, the expression of Snail, ZEB1 and Slug was measured by q-RT-PCR. Data represent mean±SD. *=significantly different from respective controls, P<0.05. (B) Transwell migration assay. Pancreatic CSCs were plated in the top chamber of the transwell and treated with EGCG (0-60 μM) for 24 h. Cells migrated to the lower chambered were fixed with methanol, stained with crystal violet and counted. Data represent mean±SD. * # or %=significantly different from respective controls, P<0.05. (C) Matrigel invasion assay. CSCs were plated onto the Matrigel-coated membrane in the top chamber of the transwell and treated with EGCG (0-60 μM) for 48 h. Cells invaded to the lower chambered were fixed with methanol, stained with crystal violet and counted. Data represent mean±SD. *, # or %=significantly different from respective controls, P<0.05. (D) Effects of EGCG on TCF-1/LEF activity. Pancreatic CSCs were transduced with TCF/LEF responsive GFP/firefly luciferase viral particles (pGreen Fire1-Gli with EF1, System Biosciences). Transduced CSCs were treated with EGCG (0-60) for 48 h and the GFP fluorescence was measured. Data represent mean±SD. *, # or **=significantly different from control, P<0.05.

FIG. 42A and FIG. 42B show graphs showing quercetin synergizes with EGCG to inhibit self-renewal capacity, invasion, migration, and TCF/LEF and Gli transcriptional activities in pancreatic CSCs. (A) Effects of EGCG and quercetin on spheroid and colony formation. Upper Panel, uercetin synergizes with EGCG to inhibit spheroid's cell viability. CSCs were seeded in suspension and treated with EGCG (0-60 μM) with or without quercetin (20 μM) for 7 days. At the end of incubation period, all the spheroids were collected and resuspended. Cell viability was measured by trypan blue assay. Data represent mean±SD. *, #, %, **, ##, or %%=significantly different from control, P<0.05. Lower panel, uercetin synergizes with EGCG to inhibit colony formation. Pancreatic CSCs were seeded in soft agar and treated with various doses of EGCG (0-60 μM) with or without quercetin (20 μM) and incubated at 4° C. for 21 days. At the end of incubation period, colonies were counted. Data represent mean±SD. *, #, %, **, ##, or %%=significantly different from control, P<0.05. (B) Effects of EGCG and quercetin on invasion and migration. Upper panel, Matrigel invasion assay. CSCs were plated onto the Matrigel-coated membrane in the top chamber of the transwell and treated with EGCG (0-60 μM) with or without quercetin (20 μM) for 48 hrs. Cells invaded to the lower chambered were fixed with methanol, stained with crystal violet and counted. Data represent mean±SD. *, #, %, **, ##, or %%=significantly different from control, P<0.05. Lower panel, Transwell migration assay. Pancreatic CSCs were plated in the top chamber of the transwell and treated with EGCG (0-60 μM) with or without quercetin (20 μM) for 48 hrs. Cells migrated to the lower chambered were fixed with methanol, stained with crystal violet and counted. Data represent mean±SD. *, #, %, **, ##, or %%=significantly different from respective controls, P<0.05. (C) Quercetin synergizes with EGCG to induce apoptosis. CSCs were seeded in suspension and treated with EGCG (0-60 μM) with or without quercetin (20 μM) for 7 days. At the end of incubation period, all the spheroids were collected. Apoptosis was measured by TUNEL assay. Data represent mean±SD. *, #, %, **, ##, or %%=significantly different from control, P<0.05. (D) Effects of EGCG and quercetin on TCF/LEF and Gli transcriptional activities. Pancreatic CSCs were transduced with either lentivirus encoding TCF/LEF responsive GFP and luciferase genes or Gli-responsive GFP and luciferase genes. Transduced CSCs were treated with EGCG (40 μM) with or without quercetin (20 μM) for 48 hrs and the luciferase activity was measured. Data represent mean±SD. *, #, or %=significantly different from respective control, P<0.05.

FIG. 43 shows graphs showing that quercetin synergizes with sulforaphane (SFN) to inhibit self-renewal capacity of pancreatic cancer CSCs. (A) Quercetin synergizes with SFN to inhibit spheroid cell viability. Pancreatic CSCs were seeded in suspension and treated with SFN (0-10 μM) with or without quercetin (20 μM) for 7 days. At the end of incubation period, all the spheroids were collected and resuspended. Cell viability was measured by trypan blue assay. Data represent mean±SD. *, &, @ or # *=significantly different from control, P<0.05. (B) Quercetin synergizes with SFN to inhibit colony formation. SFN inhibits colony formation by pancreatic CSCs. Pancreatic CSCs were seeded in soft agar and treated with various doses of SFN and incubated at 4° C. for 21 days. At the end of incubation period, colonies were counted. Data represent mean±SD. *, &, @ or #=significantly different from respective controls, P<0.05.

FIG. 44A, FIG. 44B, and FIG. 44C are graphs showing the results of treating human cancer stem cells (CSCs) and cancer cell lines from various organs with anthothecol (0-20 μM), sanguinarine (0-20 μM), or mangostine (0-20 μM) for 72 h, and measuring cell viability. Structures of the compounds are shown on the right.

FIG. 45 shows compound structures of phenyl-isothiocyanate (PITC) (A), benzyl selenocyanate (BSC) (B), and benzyl isothiocyanate (BITC) (C).

FIG. 46 is a schematic view of the nano-particle delivery system of the present invention.

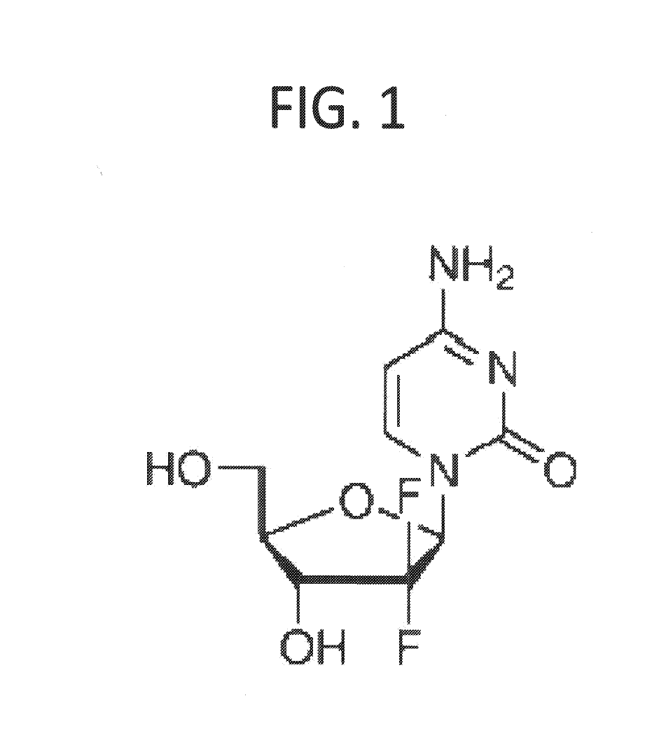

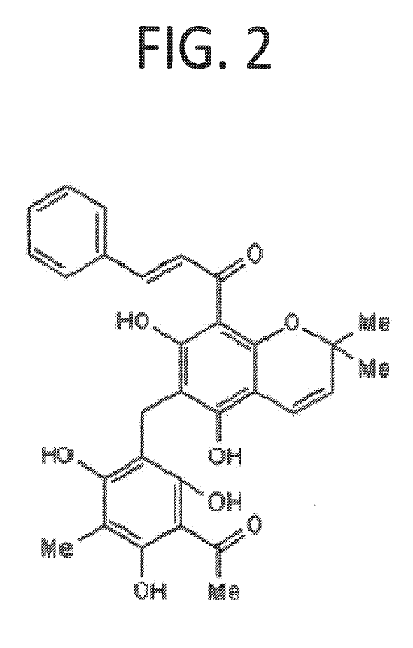

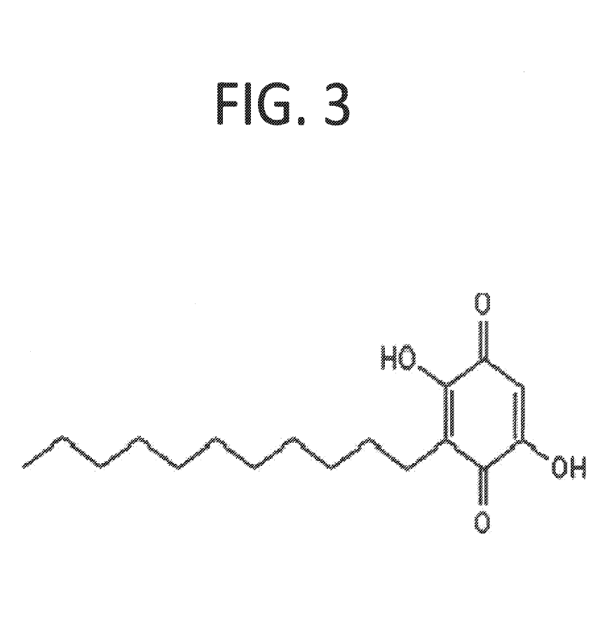

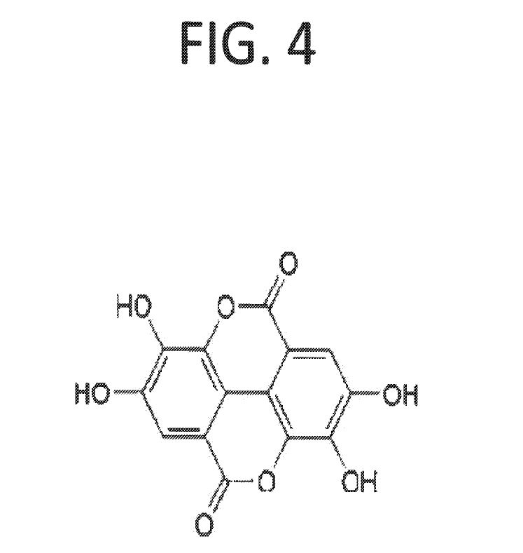

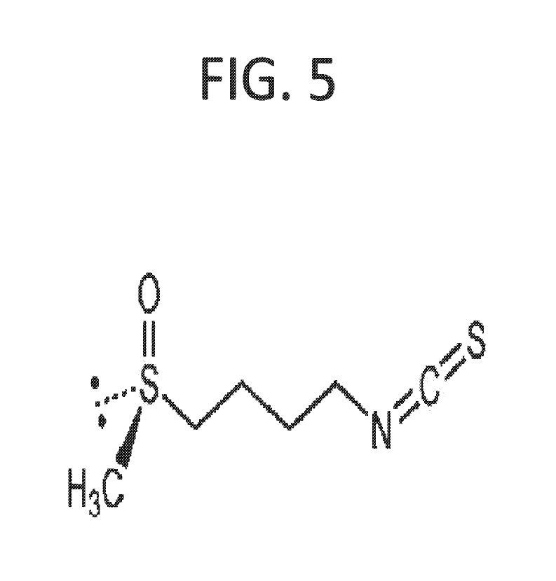

### DETAILED DESCRIPTION OF THE INVENTION

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated embodiments, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

The present invention relates generally to compositions and methods for treating cancer comprising administering to a subject in need thereof a pharmaceutically effective dose of a stem cell inhibitor.

In some embodiments, providing a therapy or “treating” cancer refers to indicia of success in the treatment, amelioration or prevention of cancer, including any objective or subjective parameter such as abatement, inhibiting metastasis, remission, diminishing of symptoms of making the disease, pathology or condition more tolerable to the patient, slowing the rate of degeneration or decline, making the final point of degeneration less debilitating, or improving a patient's physical or mental well-being. Those in need of treatment include those already with cancer as well as those prone to have cancer or in those in whom cancer is to be prevented.

In general, a pharmaceutically effective dose is meant an amount that produces the desired effect for which it is administered. The exact amount will depend on a variety of factors such as the purpose of the treatment, composition or dosage form, the selected mode of administration, the age and general condition of the individual being treated, the severity of the individual's condition, and other factors known to the prescribing physician and will be ascertainable by a person skilled in the art using known methods and techniques for determining effective doses. In some embodiments, a pharmaceutically effective dose results in a cellular concentration of the drug of from about 1 nM to 30 μM. In some embodiments, a pharmaceutically effective dose results in a cellular concentration of the drug of from about 50 nM to about 10 μM, from about 50 nM to about 1 μM, from about 100 nM to about 1 μM, or from about 100 nM to about 500 nM. In some embodiments, a pharmaceutically effective dose includes between about 0.1 mg/kg/day to about 300 mg/kg/day. In some embodiments, a pharmaceutically effective dose includes between about 1.0 μg/kg/day to about 50 mg/kg/day.

The present invention also relates to methods of inhibiting the growth of cancer stem cells comprising administering to a subject in need thereof a pharmaceutically effective dose of a stem cell inhibitor.

The present invention also relates to methods of inhibiting the growth of cancer stem cells comprising contacting cancer stem cells with an effective dose of a stem cell inhibitor.

In one embodiment, the present disclosure provides a method of enhancing the biological effects of a chemotherapeutic drug on cancer cells comprising administering to a subject in need thereof along with a chemotherapeutic drug a pharmaceutically effective dose of a stem cell inhibitor.

In one embodiment, the present invention relates to methods of treating pancreatic cancer using stem cell inhibitors.

As described herein, there are certain natural products, including rottlerin, embelin, ellagic acid, and sulforaphane, which can act as cancer stem cell inhibitors and inhibit the growth of cancer stem cells and cancer cells. These products have the advantages of being non-toxic and bioavailable and may inhibit the growth of pancreatic and other cancers and the growth of cancer stem cells. Without being bound by theory, in some embodiments it is believed that these compounds inhibit oncogenic PI3/AKT and ERK pathways, and thus can be used as cancer preventive agents. In some embodiments, sulforaphane inhibits the growth of pancreatic cancer stem cells. In some embodiments, sulforaphane blocks pancreatic cancer progression in an animal model, such as KrasG12D mice. In some embodiments, sulforaphane enhances the biological effects of gemcitabine and lapatinib on cancer stem cells. In some embodiments, sulforaphane enhances the biological effects of gemcitabine and lapatinib on pancreatic cancer stem cells.

Cancer stem cells (CSCs) have been proposed recently to be the cause cancer initiation, progression and chemotherapy failure. CSCs also demonstrate upregulation of signaling pathways such as sonic hedgehog (Shh), Wnt and Notch. Regulation of CSCs by non-toxic agents could be considered as a strategy for the treatment and/or prevention of cancer.

In one embodiment, the present invention provides a method of treating cancer comprising administering to a subject in need a pharmaceutically effective dose of a stem cell inhibitor. In certain embodiments, the stem cell inhibitor may comprise rottlerin, embelin, ellagic acid, sulforaphane, resveratrol, honokiol, curcumin, diallyltrisulfide, benzyl isothiocyanate, quercetin, epigallocatechin gallate (EGCG), SAHA, m-Carboxycinnamic acid bis-hydroxamine, and/or MS-275. In certain embodiments, the stem cell inhibitor may comprise epigenetic regulators and agents that modify histones and DNA such as SAHA/vornostat, m-Carboycinnamic acid bis-hydroxamine, MS-275, and demathylating agent such as 5-aza-2′-deoxycytidine.

Rottlerin is a polyphenolic compound derived from Mallotus philipinensis (Euphorbiaceae). It is widely used as an inhibitor of PKCδ due to the competition between rottlerin and ATP, which plays a crucial role in apoptosis, cell migration and cytoskeleton remodeling. These cellular functions are important regulators of tumor progression and metastasis. In addition to inhibiting PKCδ, rottlerin targets mitochondria to induce apoptosis. Rottlerin causes uncoupling of mitochondrial respiration from oxidative phosphorylation and a collapse of mitochondrial membrane potential in several cell types. Rottlerin has been shown to induce apoptosis in various cancer cells, including prostate, colon, pancreatic and lung cancer cells, chronic leukemia, and multiple myeloma cells. Rottlerin has been shown to inhibit cancer cell migration. Rottlerin has not previously been used to inhibit CSC self-renewal and tumor growth. Furthermore, there are no previous studies demonstrating the regulation of CSCs by rottlerin, and whether rottlerin can inhibit sonic hedgehog, Wnt and Notch pathways.

Embelin is a polyphenolic compound derived from the fruit of Embelia ribes Burm plant (Myrsinaceae). Embelin is a cell-permeable, non-peptide inhibitor of X-linked inhibitor of apoptosis (XIAP); binds to the BIR3 domain, preventing XIAP interaction with caspase-9 and Smac. It inhibits cell growth, induces apoptosis and activates caspase-9 in cancer cells. Embelin possesses wide spectrum of biological activities with strong inhibition of nuclear factor kappa B and downstream antiapoptotic genes. These cellular functions are important regulators of tumor progression and metastasis. Embelin has been shown to induce apoptosis in various cancer cells, including prostate, colon, pancreatic and lung cancer cells, chronic leukemia, and multiple myeloma cells. Embelin has not previously been used to inhibit CSC self-renewal and tumor growth. Furthermore, there are no previous studies demonstrating the regulation of CSCs by embelin, and whether embelin can inhibit Sonic hedgehog, Notch and Wnt pathways.

Ellagic acid is a compound derived from berries and nuts, it is a hydrolytic product of ellagitannins.

Sulforaphane (SFN) is a compound found in cruciferous vegetables. It is shown herein that sulforaphane inhibits the growth of human pancreatic cancer cells and pancreatic cancer stem cells. Furthermore, SFN also inhibits the growth of pancreatic cancer progression in KrasG12D mice. In some embodiments of the invention, quercetin can enhance the inhibitory effects of sulforaphane on cancer stem cells, such as pancreatic cancer stem cells.

In some embodiments, one or more of rottlerin, embelin, ellagic acid, and sulforaphane can be used to kill cancer cells and inhibit cancer stem cell growth by targeting sonic hedgehog, Notch and Wnt pathways. Therefore, these compounds may be used to target cancer stem cells and kill them. They are non-toxic and bioavailable and, since these compounds are derived from plant/natural sources, they may be given to patients safely. In some embodiments, these compounds may inhibit the self-renewal capacity of CSCs by inhibiting pluripotency maintaining factors and Notch, Wnt and Shh pathways. Thus, these compounds may be a potent biologic inhibitor of cancer stem cells and can be used to treat and/or prevent cancer. These compounds may also modulate the expression of genes and pathways known to play roles in the carcinogenesis process and, therefore, may be used as agents for chemoprevention and/or therapy against cancer.

In some embodiments, the compounds may inhibit survival pathways such as AKT and MAPK/ERK, which can be activated by oncogenic Kras. In some embodiments, one or more of rottlerin, embelin and ellagic acid inhibit pathways downstream of Kras to treat or prevent cancer in pancreatic cancer subjects.

In some embodiments, sulforaphane enhances the biological effects of gemcitabine and lapatinib on pancreatic cancer stem cells.

In some embodiments, these agents can be used in conjunction with other cancer therapies. In some embodiments, one or more of the compounds are used with other anticancer drugs, such as, for example gemcitabine and lapatinib, irradiation to sensitize cancer stem cells, and/or surgical intervention. Other anticancer drugs that can be combined with the compounds as described herein include, for example, Abraxane, Aldara, Alimta, Aprepitant, Arimidex, Aromasin, Arranon, Arsenic Trioxide, Avastin, Bevacizumab, Bexarotene, Bortezomib, Cetuximab, Clofarabine, Clofarex, Clolar, Dacogen, Dasatinib, Ellence, Eloxatin, Emend, Erlotinib, Faslodex, Femara, Fulvestrant, Gefitinib, Gemtuzumab Ozogamicin, Gemzar, Gleevec, Herceptin, Hycamtin, Imatinib Mesylate, Iressa, Kepivance, Lenalidomide, Levulan, Methazolastone, Mylosar, Mylotarg, Nanoparticle Paclitaxel, Nelarabine, Nexavar, Nolvadex, Oncaspar, Oxaliplatin, Paclitaxel, Paclitaxel Albumin-stabilized Nanoparticle Formulation, Palifermin, Panitumumab, Pegaspargase, Pemetrexed Disodium, Platinol-AQ, Platinol, Revlimid, Rituxan, Sclerosol Intrapleural Aerosol, Sorafenib Tosylate, Sprycel, Sunitinib Malate, Sutent, Synovir, Tamoxifen, Tarceva, Targretin, Taxol, Taxotere, Temodar, Temozolomide, Thalomid, Thalidomide, Topotecan Hydrochloride, Trastuzumab, Trisenox, Vectibix, Velcade, Vidaza, Vorinostat, Xeloda, Zoledronic Acid, Zolinza, Zometa, doxorubicin, adriamycin, bleomycin, daunorubicin, dactinomycin, epirubicin, idarubicin, mitoxantrone, valrubicin, hydroxyurea, mitomycin, fluorouracil, 5-FU, methotrexate, floxuridine, interferon alpha-2b, glutamic acid, plicamycin, 6-thioguanine, aminopterin, pemetrexed, raltitrexed, cladribine, clofarabine, fludarabine, mercaptopurine, pentostatin, capecitabine, cytarabine, carmustine, BCNU, lomustine, CCNU, cytosine arabinoside, cyclophosphamide, estramustine, hydroxyurea, procarbazine, mitomycin, busulfan, medroxyprogesterone, estramustine phosphate sodium, ethinyl estradiol, estradiol, megestrol acetate, methyltestosterone, diethylstilbestrol diphosphate, chlorotrianisene, testolactone, mephalen, mechlorethamine, chlorambucil, chlormethine, ifosfamide, bethamethasone sodium phosphate, dicarbazine, asparaginase, mitotane, vincristine, vinblastine, etoposide, teniposide, Topotecan, IFN-gamma, irinotecan, campto, irinotecan analogs, carmustine, fotemustine, lomustine, streptozocin, carboplatin, oxaliplatin, BBR3464, busulfan, dacarbazine, mechlorethamine, procarbazine, thioTEPA, uramustine, vindesine, vinorelbine, alemtuzumab, tositumomab, methyl aminolevulinate, porfimer, verteporfin, lapatinib, nilotinib, vandetanib, ZD6474, alitretinoin, altretamine, amsacrine, anagrelide, denileukin diftitox, estramustine, hydroxycarbamide, masoprocol, mitotane, tretinoin, or other anticancer drugs, including, for example, antibiotic derivatives, cytotoxic agents, angiogenesis inhibitors, hormones or hormone derivatives, nitrogen mustards and derivatives, steroids and combinations, and antimetbolites. Other chemotherapeutic drugs include Notch inhibitor, TGFbeta inhibitor, TCF/LEF inhibitor, Nanog inhibitor, AKT inhibitor, FLT3 kinase inhibitor, PI3 Kinase inhibitor, PI3 kinase/mTOR (dual inhibitor), PI3K/AKT pathway inhibitor, Hedgehog pathway inhibitor, Gli inhibitor, Smoothened inhibitor, JAK/STAT pathway inhibitor, Ras/MEK/ERK pathway inhibitor, and BRAF inhibitor. In further particular aspects of the invention, an anticancer drug comprises two or more of the foregoing anticancer drugs.

Suitable cancers which can be treated by inhibiting cancer stem cells using the compositions and methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); cancers of the lymphomas (hodgkin's disease and non-hodgkin's lymphoma), multiple myeloma, and leukemias (lymphocytic leukemia; myeloid leukemia; monocytic leukemia; and other leukemias).

Other cancers, classified by histological type, that may be treated include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Branchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malignant melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovari, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In some embodiments, the cancer to be treated and the cancer stem cells to be inhibited are from cancers selected from the group consisting of breast cancer, prostrate cancer, brain cancer, lung cancer, mesothelioma, melanoma, multiple myeloma, colon cancer, kidney cancer, ovarian cancer, pancreatic cancer, leukemia, and lymphoma.

The “subject” of the cancer treatment methods and compositions according to the invention includes, but is not limited to, a mammal, such as a human, mouse, rat, pig, cow, dog, cat, or horse. In one embodiment, the subject is a human or person.

In the compositions and methods of the invention, cancer stem cell inhibitors can be administered by various routes of administration, including, for example, intraarterial administration, epicutaneous administration, eye drops, intranasal administration, intragastric administration (e.g., gastric tube), intracardiac administration, subcutaneous administration, intraosseous infusion, intrathecal administration, transmucosal administration, epidural administration, insufflation, oral administration (e.g., buccal or sublingual administration), oral ingestion, anal administration, inhalation administration (e.g., via aerosol), intraperitoneal administration, intravenous administration, transdermal administration, intradermal administration, subdermal administration, intramuscular administration, intrauterine administration, vaginal administration, administration into a body cavity, surgical administration (e.g., at the location of a tumor or internal injury), administration into the lumen or parenchyma of an organ, or other topical, enteral, mucosal, parenteral administration, or other method or any combination of the forgoing as would be known to one of ordinary skill in the art (see, for example, Remington's Pharmaceutical Sciences, 18th Ed. Mack Printing Company, 1990, incorporated herein by reference).

Targeted drug delivery, sometimes called smart drug delivery, is a method of delivering medication to a patient in a manner that increases the concentration of the medication in some parts of the body relative to others. The goal of a targeted drug delivery system is to prolong, localize, target and have a protected drug interaction with the diseased tissue. The conventional drug delivery system is the absorption of the drug across a biological membrane, whereas the targeted release system is when the drug is released in a dosage form. The advantages to the targeted release system is the reduction in the frequency of the dosages taken by the patient, having a more uniform effect of the drug, reduction of drug side effects, and reduced fluctuation in circulating drug levels. Drugs can be delivered using liposomes, micelles and dendrimers, polymers, biodegradable particles, and artificial DNA nanostructure. Particles (diameter 80 to 600 nM) comprised of the polymer poly(lactic-co-glycolic acid) (PLGA) are widely studied as therapeutic delivery vehicles because they are biodegradable and biocompatible. PLGA particles also offer considerable flexibility in choosing a route of delivery because they have proven to be effective when injected intramuscularly, when delivered via inhalation, and have been recently indicated for oral delivery of drugs and antigens.

Suitable compositions and dosage forms also include tablets, capsules, nanoparticles, caplets, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, and the like.

Many engineered multifunctional nanoparticles can be used as pharmaceutical drug carriers, including nano-emulsions, dendrimers, nano-gold, polymers, carbohydrates, liposomes, drug-carrier conjugates, antibody-drug complexes, and magnetic nanoparticles, are primarily based on unique assemblies of synthetic or biological components, including but not limited to synthetic polymers, metal ions, oils, and lipids as their building blocks.

It is noted that the carrier is a key component of the composition claimed in the present application. It has direct impact on the efficacy of the delivered active ingredient.

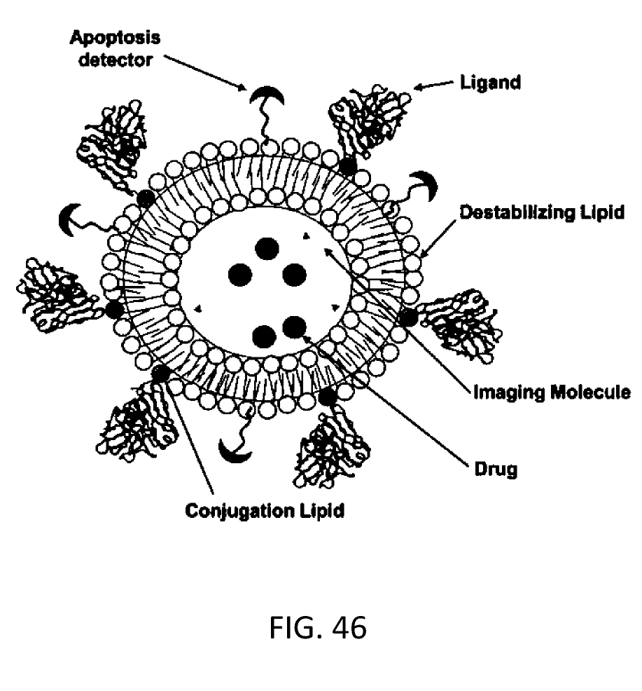

In reference to FIG. 46 below, it has shown a schematic view of one design principle of a nanoparticle for targeted delivery of therapeutics. This nano-assembly includes imaging molecules, a payload of drugs, ligands for site-specific targeting, and a destabilizing lipid that allows for on-demand drug release at the desired site, as well as sensors that probe the efficacy of the drug in real time. Some widely examined nano-carriers aimed at delivering DNA, pharmaceuticals, and/or imaging agents include dendrimers, nano-gold shells, nano-emulsions, drug-polymer conjugates, drug-antibody conjugates and quantum dots. Each of these is based on unique properties of the structural components used in fabricating the delivery vehicle and relies on self-assembly of the structural motifs, while accommodating the pharmaceutical agent and the targeting ligand. A limited number of the drug-loaded nanoparticles are successful for their clinical applications. An important parameter of the delivery vehicle pertains to low or no toxicity of the carrier itself either in vivo or in the environment as a by-product.

Oral dosage forms are preferred for those therapeutic agents that are orally active, and include tablets, capsules, caplets, solutions, suspensions and/or syrups, and may also comprise a plurality of granules, beads, powders or pellets that may or may not be encapsulated. Such dosage forms can be prepared using conventional methods known to those in the field of pharmaceutical formulation and described in the pertinent texts, e.g., in Remington: The Science and Practice of Pharmacy, 20th Edition, Gennaro, A. R., Ed. (Lippincott, Williams and Wilkins, 2000).

Tablets and capsules represent the most convenient oral dosage forms, in which case solid pharmaceutical carriers are employed. Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), tablets prepared for oral administration will generally contain other materials such as binders, diluents, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts, e.g., in Remington, supra. Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Parenteral administration, if used, is generally characterized by injection, including intramuscular, intraperitoneal, intravenous (IV) and subcutaneous injection. Injectable formulations can be prepared in conventional forms, either as liquid solutions or suspensions, solid forms suitable for solution or suspension in liquid prior to injection, or as emulsions. In some embodiments, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. In some embodiments, the formulation for parenteral administration is a controlled release formulation, such as delayed or sustained release.

Any of the active agents may be administered in the form of a salt, ester, amide, prodrug, active metabolite, derivative, or the like, provided that the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base using conventional methodology, and involves reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are salts prepared with organic acids. Conversely, preparation of basic salts of acid moieties which may be present on an active agent are prepared in a similar manner using a pharmaceutically acceptable base such as sodium hydroxide, potassium hydroxide, ammonium hydroxide, calcium hydroxide, trimethylamine, or the like. Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

In addition, it is noted that various combinations of a plurality of the cancer stem cell inhibitors may provide a better treatment efficacy. For instance, the inhibitors including sulforaphane, resveratrol, mangostine, honokiol, diallyltrisulphide and gemcitabine may be employed in various combination to provide a synergistic treating effects. The possible combinations include sulforaphane, resveratrol, mangostine, honokiol and diallyltrisulphide; sulphoraphane, resveratrol and gemcitabine; mangostine, resveratrol and gemcitabine; honokiol, resveratrol and gemcitabine and the like.

Moreover, as for the above described combination, they may be administered to subjects in specific doses as follows,

Combination I: Sulforaphane (about 0.1 mg to 300 mg/kg/day), resveratrol (about 0.1 mg to 300 mg/kg/day), mangostine (about 0.1 mg to 300 mg/kg/day), honokiol (about 0.1 mg to 300 mg/kg/day) and diallyltrisulphide (about 0.1 mg to 300 mg/kg/day). The relative ratio of the sulforaphane, resveratrol, mangostine, honokiol, and diallyltrisulphide will be about 1 to 30%, 1 to 30%, 1 to 30%, 1 to 30%, and 1 to 30%, respectively.

Combination II: Sulphoraphane (about 0.1 mg to 500 mg/kg/day), resveratrol (about 0.1 mg to 500 mg/kg/day) and gemcitabine (about 1.0 μg/kg/day to about 100 mg/kg/day). The relative ratio of the sulforaphane, resveratrol, and gemcitabine will be about 1 to 45%, 1 to 45%, and 0.1 to 30%, respectively.

Combination III: Mangostine (about 0.1 mg to 500 mg/kg/day), resveratrol (about 0.1 mg to 500 mg/kg/day) and gemcitabine (about 1.0 μg/kg/day to about 100 mg/kg/day). The relative ratio of the mangostine resveratrol, and gemcitabine will be about 1 to 45%, 1 to 45%, and 0.1 to 30%, respectively.

Combination IV: Honokiol (about 0.1 mg to 500 mg/kg/day), resveratrol (about 0.1 mg to 500 mg/kg/day) and gemcitabine (about 1.0 μg/kg/day to about 100 mg/kg/day). The relative ratio of the honokiol, resveratrol, and gemcitabine will be about 1 to 45%, 1 to 45%, and 0.1 to 30%, respectively.

To facilitate a better understanding of the present invention, the following examples of certain aspects of some embodiments are given. In no way should the following examples be read to limit, or define, the entire scope of the invention.

### Example 1

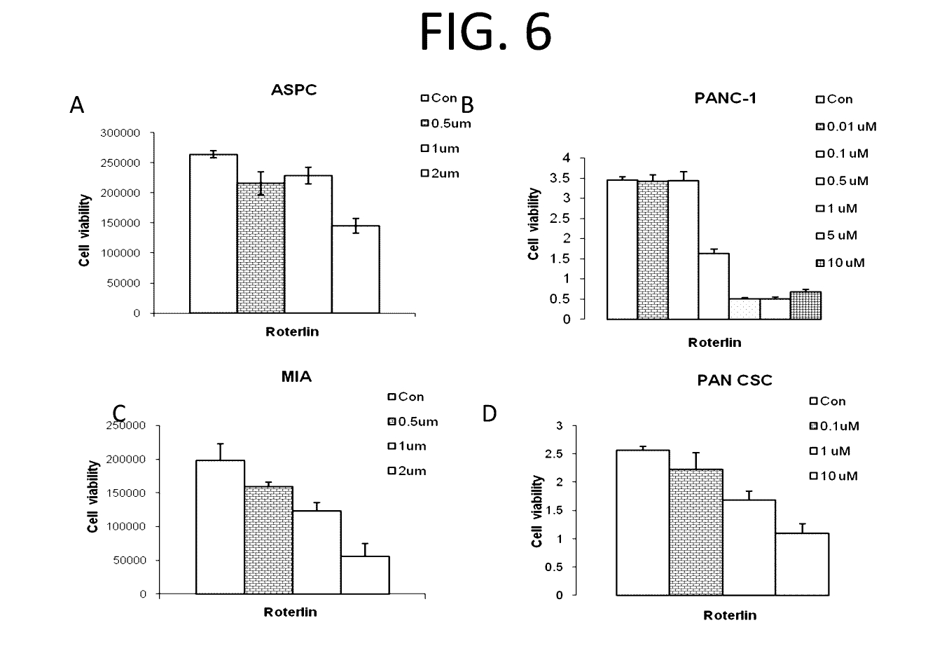

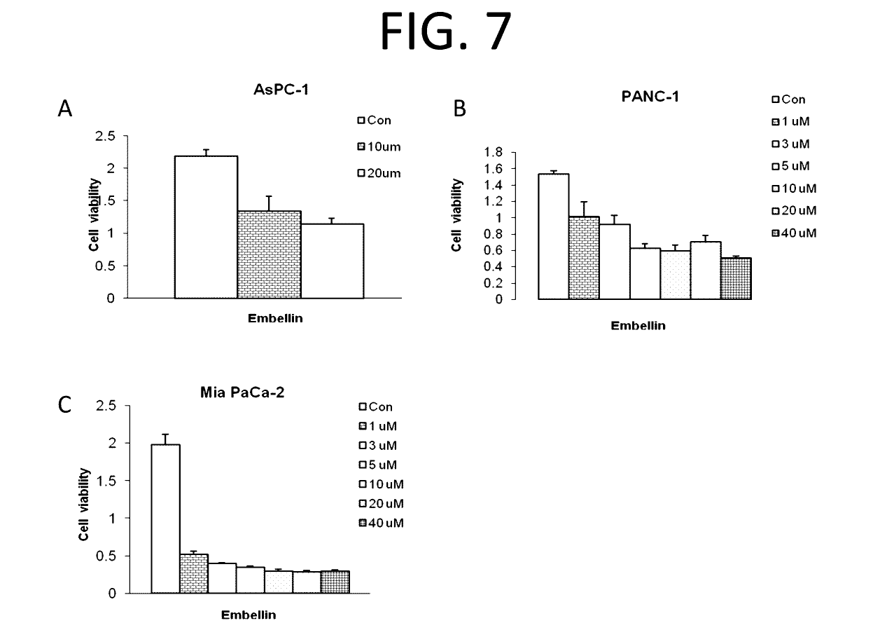

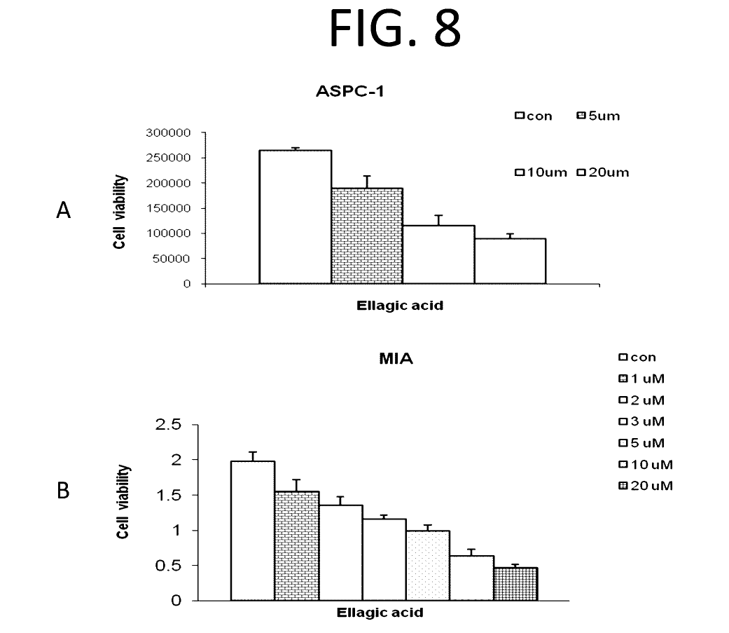

The effects of rottlerin, embelin, and ellagic acid on the growth of human pancreatic cancer cells and cancer stem cells were studied. Pancreatic cancer cells AsPC-1, PANC-1, and MIA PaCa-2 and pancreatic cancer stem cells were treated with rottlerin for 3 days and then cell viability was measured by XTT assay. Pancreatic cancer cells AsPC-1, PANC-1, and MIA PaCa-2 were treated with embelin for 3 days and cell viability was measured by XTT assay. Pancreatic cancer cells AsPC-1 and MIA PaCa-2 were treated with ellagic acid for 3 days and cell viability was measured by XTT assay. The results of these studies are illustrated in FIGS. 6-8.

### Example 2

### Embelin Inhibits Growth and Induces Apoptosis in Prostate CSCs

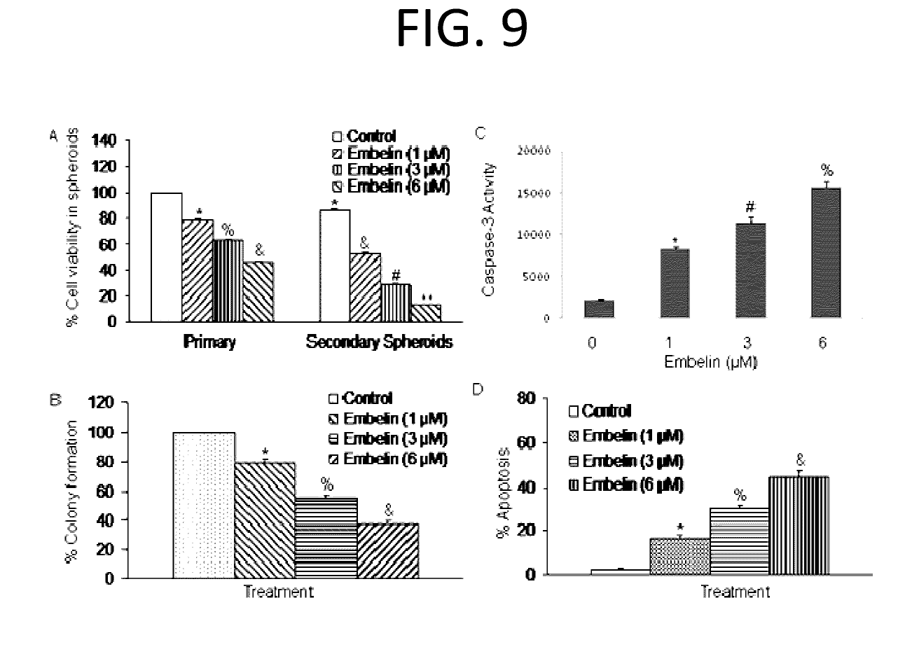

The effects of embelin on growth of prostate CSCs was studied by measuring cell viability and colony formation, as shown in FIGS. 9A and 9B. Embelin inhibited the size of primary and secondary spheroids in suspension (data not shown), and cell viability in spheroids and colony formation in soft agar. Since embelin inhibited the self-renewal capacity of CSCs in vitro, effects of embelin on caspase-3 activity and apoptosis were examined, as shown in FIGS. 9C and 9D. Embelin may induce caspase-3 activity and apoptosis. The data suggests that embelin can inhibit self-renewal capacity of CSCs.

### Embelin Inhibits the Expression of Bcl-2, Survivin and XIAP in Prostate CSCs.

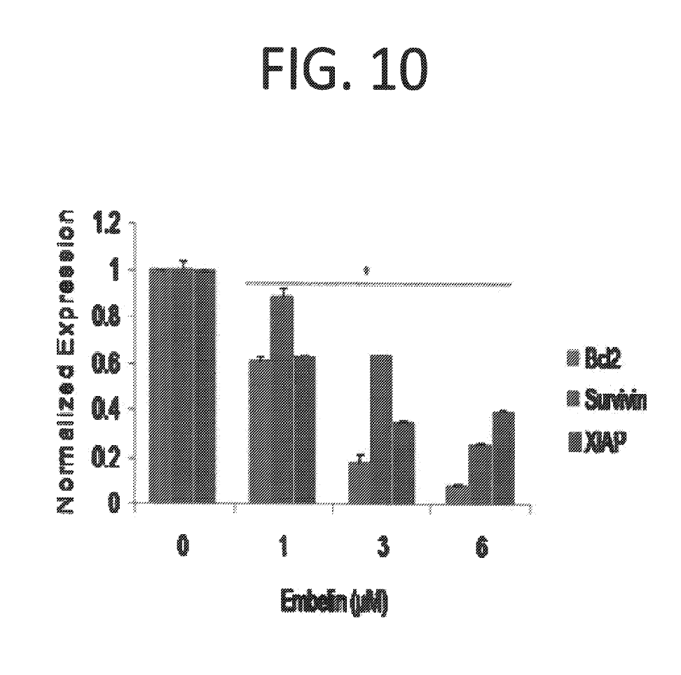

Since IAPs and Bcl-2 family members may play major roles in regulation of cell survival and apoptosis, the effects of embelin on the expression of Bcl-2, survivin and XIAP were examined. Embelin inhibited the expression of Bcl-2, survivin and XIAP, as shown in FIG. 10. The data suggests that embelin can regulate self-renewal capacity of prostate CSCs through inhibition of Bcl-2 and IAPs.

### Embelin Inhibits the Expression of Nanog and Oct3/4.

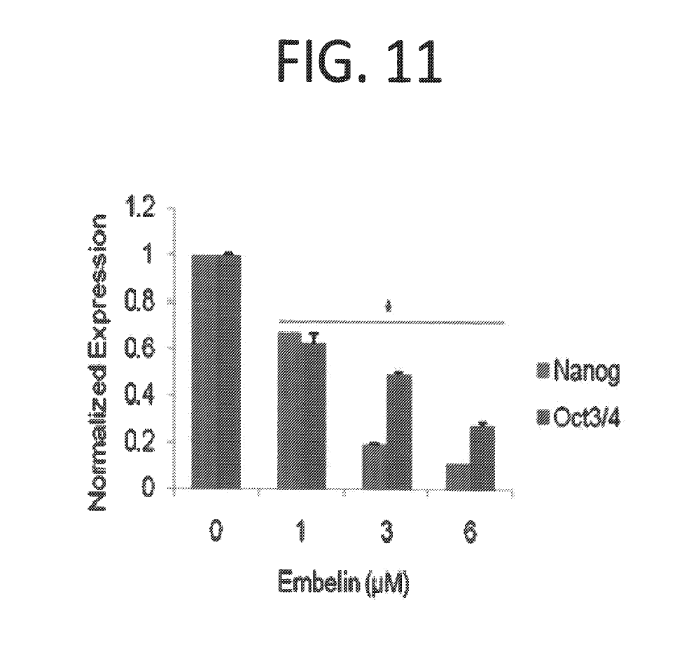

Since Nanog and Oct3/4 may be highly expressed in CSCs, and may be required for maintaining pluripotency, the effects of embelin on the expression of these genes in human prostate CSCs were examined, as shown in FIG. 11. Embelin inhibited the expression of Nanog and Oct3/4. The data suggests that embelin inhibits the factors required for maintaining pluripotency in prostate CSCs.

### Embelin Inhibits Shh Signaling Pathway.

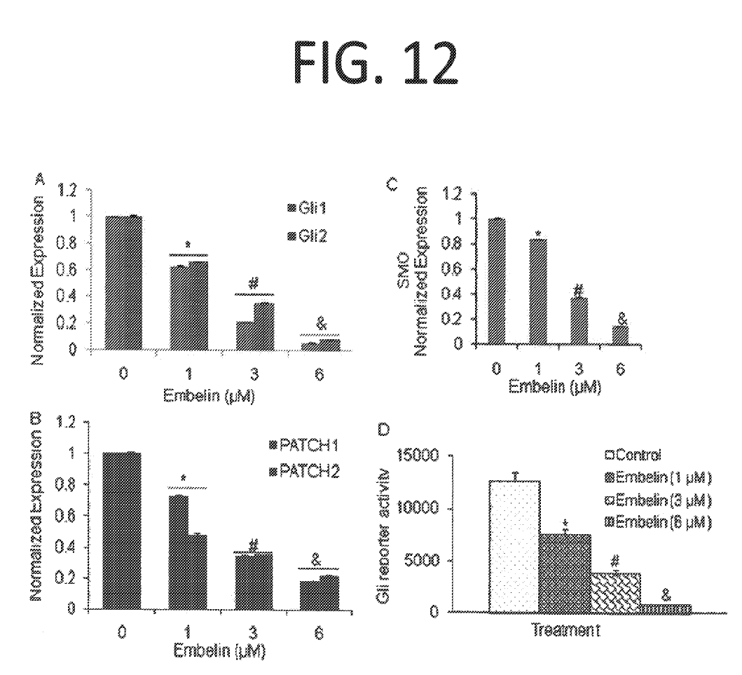

The effects of embelin on Shh pathway by measuring the expression of Shh receptors (Patched-1, Patched-2 and Smoothened) and effectors (Gli1 and Gli2) by qRT-PCR were examined. Embelin inhibited the expression of Gli1, Gli2, Patched-1, Patched-2, and smoothened (SMO), as shown in FIGS. 12A-C. Embelin inhibited Ptch 1 and Ptch 2 because they are downstream targets of Gli. Since Gli transcription factor may mediate the effects of Shh which play important roles in maintaining stemness and tumorigenesis, the Gli transcriptional activity was measured, as shown in FIG. 12D. As shown in FIG. 12D, embelin inhibited Gli transcriptional activity in a dose-dependent manner. The data suggests that embelin can inhibit prostate CSC characteristics by inhibiting Shh pathway which has been shown to play an important role in maintaining stemness.

### Embelin Inhibits Markers of Epithelial-Mesenchymal Transition (EMT) in Human Prostate CSCs.

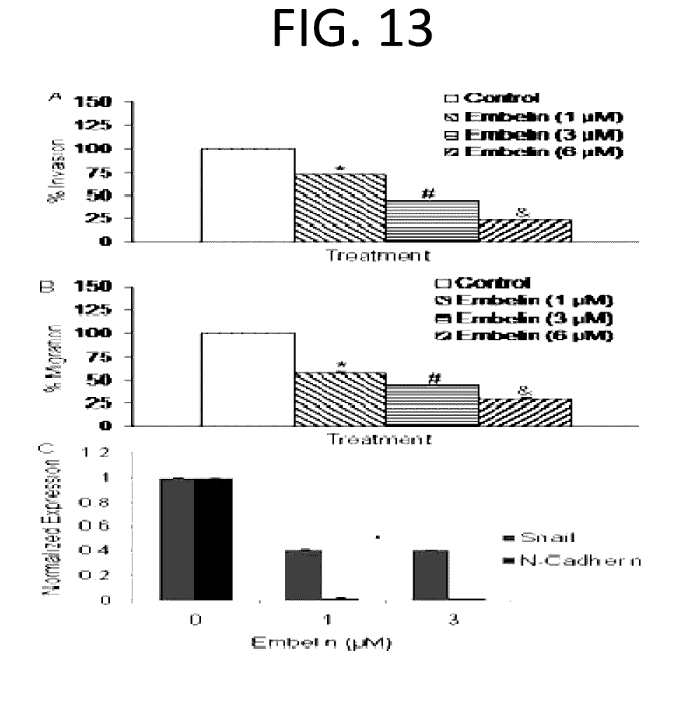

Recent studies revealed that there is a direct link between the EMT program and the gain of epithelial stem cell properties. The effects of embelin on invasion, migration and the expression of mesenchymal marker N-cadherin and EMT transcription factor Snail in prostate CSCs were examined, as shown in FIG. 13. Embelin inhibited CSC invasion, and migration, and also the expression of Snail and N-cadherin. The data suggests that embelin can inhibit or reverse EMT which is required for early metastasis.

### Example 3

### Rottlerin Inhibits Growth of Prostate Cancer Stem Cells

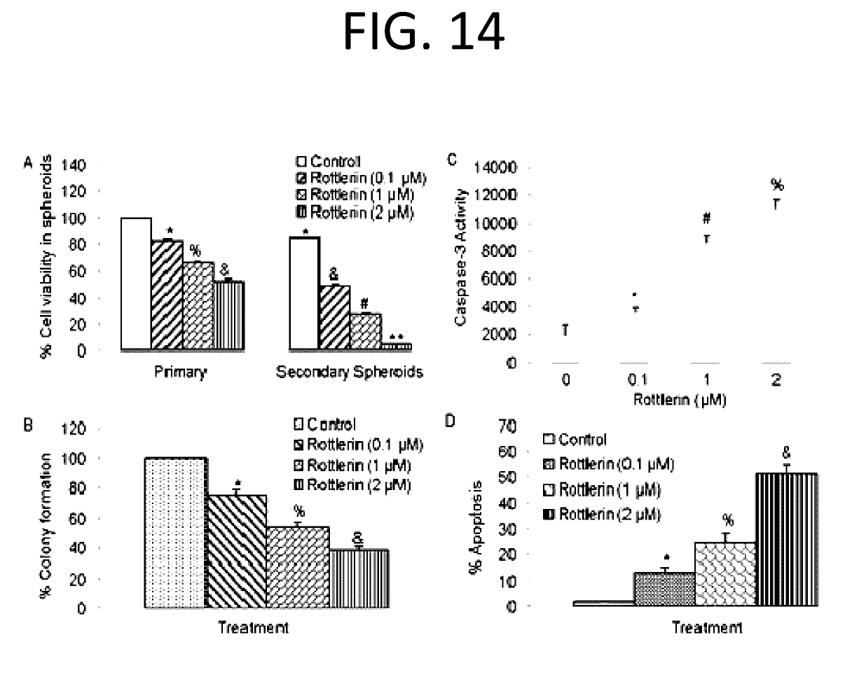

The effects of rottlerin on growth of prostate CSCs by measuring cell viability and colony formation were studied, as shown in FIGS. 14A and 14B. Rottlerin inhibited the size of primary and secondary spheroids in suspension (data not shown), and cell viability in spheroids and colony formation in soft agar. Since rottlerin inhibited the self-renewal capacity of CSCs in vitro, the effects of rottlerin on caspase-3 activity and apoptosis were examined, as shown in FIGS. 14C and 14D. Rottlerin may induce caspase-3 activity and apoptosis. The data suggests that rottlerin can inhibit self-renewal capacity of CSCs.

### Rottlerin Inhibits the Expression of Survivin, XIAP, Bcl-2 and Bcl-X_{L} in Prostate CSCs.

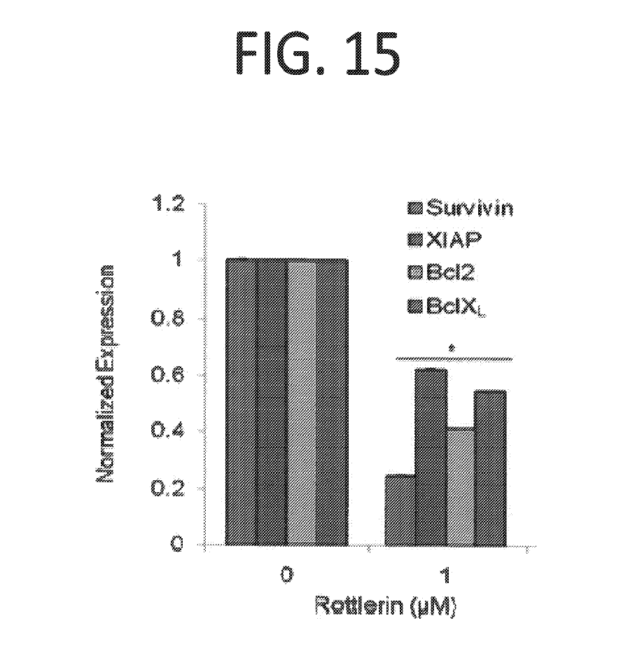

Since IAPs and Bcl-2 family members may play major roles in regulation of cell survival and apoptosis, the effects of rottlerin on the expression of survivin, XIAP, Bcl-2 and Bcl-X_{L} were examined. Rottlerin inhibited the expression of survivin, XIAP, Bcl-2 and Bcl-X_{L}, as shown in FIG. 15. The data suggests that rottlerin can inhibit survival and induce apoptosis in prostate CSCs through inhibition of IAPs and anti-apoptotic Bcl-2 and Bcl-X_{L}.

Rottlerin Inhibits the Expression of cMyc, Nanog, Oct3/4 and Sox-2.

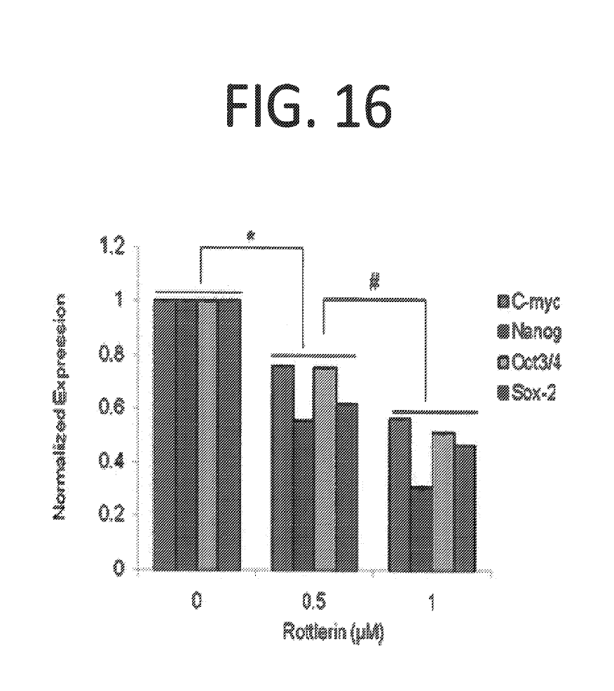

Since cMyc, Nanog, Oct3/4 and Sox-2 may be highly expressed in CSCs, and may be required for maintaining pluripotency, the effects of rottlerin on the expression of these genes in human prostate CSCs were examined, as shown in FIG. 16. Rottlerin inhibited the expression of cMyc, Nanog, Oct3/4 and Sox-2. The data suggests that rottlerin inhibits the factors required for maintaining pluripotency in prostate CSCs.

### Rottlerin Inhibits Shh, Notch and TGFβ Signaling Pathways.

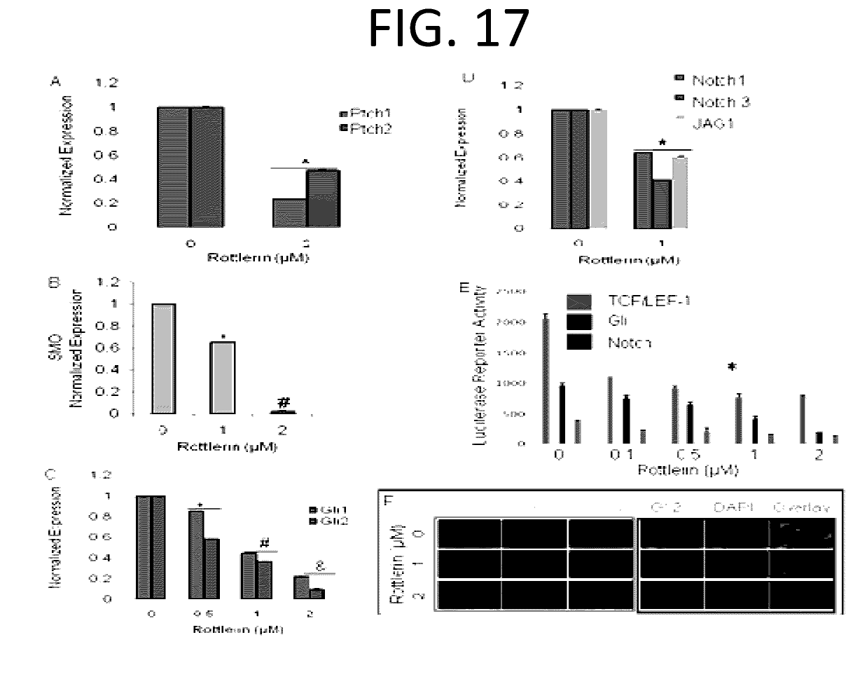

The effects of rottlerin on the Shh pathway were examined by measuring the expression of Shh receptors (Patched-1, Patched-2 and Smoothened) and effectors (Gli1 and Gli2) by qRT-PCR. Rottlerin inhibited the expression of Patched-1, Patched-2, SMO, Gli1 and Gli2 were examined, as shown in FIGS. 17A-C. Rottlerin also inhibited the expression of Notch1, Notch3 and JAG1. Shh, Notch and TGFβ signaling pathways interact together and play important roles in maintaining stemness and tumorigenesis, therefore the TCF/LEF1, Gli and Notch reporter activities were measured, as shown in FIG. 17E. Rottlerin inhibited the TCF/LEF1, Gli1 and Notch responsive reporter activities in a dose-dependent manner. Ptch 1 and 2 are the downstream targets of Gli transcription factor. Rottlerin also inhibited the nuclear expression of constitutively active Gli1 and Gli2 as measured by IFC, as shown in FIG. 17F. The data suggests that rottlerin can inhibit prostate CSC characteristics by inhibiting Shh, Notch and TGFβ pathways which have been shown to interact together.

### Rottlerin Inhibits Growth of Pancreatic Cancer Stem Cells.

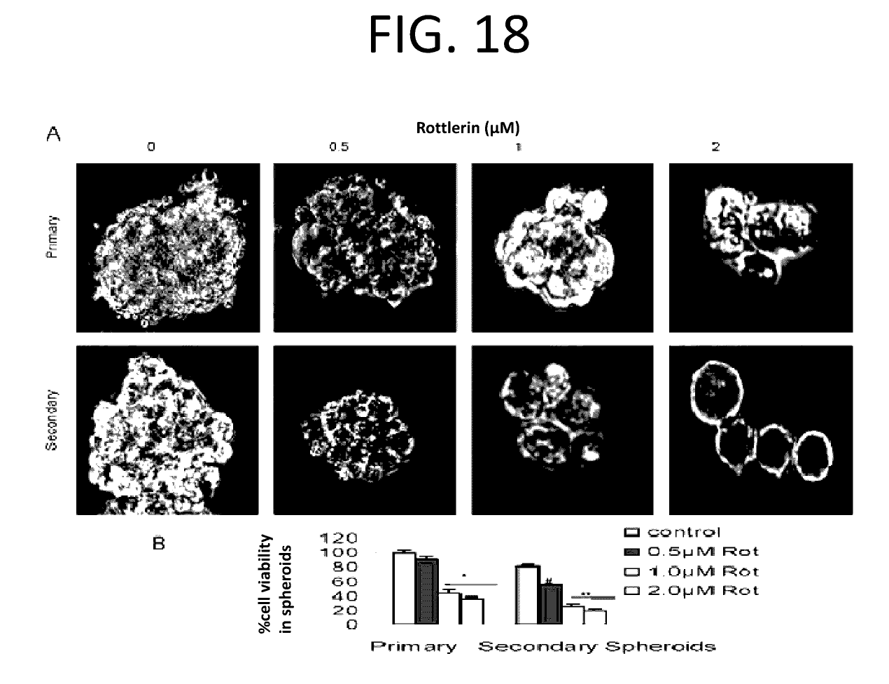

The effects of rottlerin on growth of pancreatic CSCs isolated from human pancreatic tumors by growing them in spheroids and measuring their cell viability in spheroids were examined, as shown in FIG. 18. Rottlerin inhibited the size of primary and secondary spheroids in suspension, and cell viability of spheroids, as shown in FIGS. 18A and 18B. The data suggests that rottlerin is effective in inhibiting the growth of pancreatic CSCs.

Rottlerin Inhibits the Expression of cMyc, Nanog, Oct-4 and Sox-2 in Pancreatic CSCs.

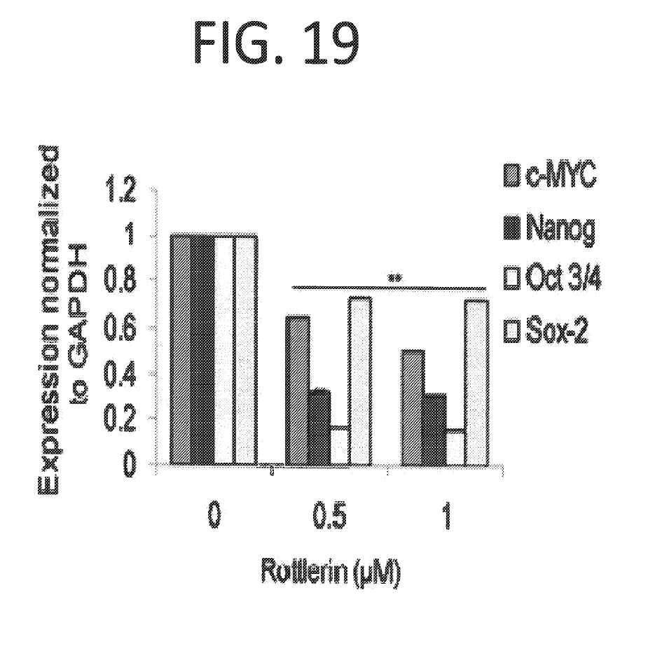

Since transcription factors cMyc, Nanog, Sox-2, and Oct-4 may be highly expressed in cancer stem cells and may be required for maintaining pluripotency, the effects of rottlerin on the expression of cMyc, Nanog, Sox-2, and Oct-4 in human pancreatic CSCs were examined. Rottlerin inhibited the expression of Nanog, Sox-2 and cMyc as measured by qRT-PCR, as shown in FIG. 19. The data suggests that rottlerin inhibits the factors required for maintaining pluripotency in pancreatic CSCs.

### Rottlerin Inhibits Hedgehog Signaling Pathway.

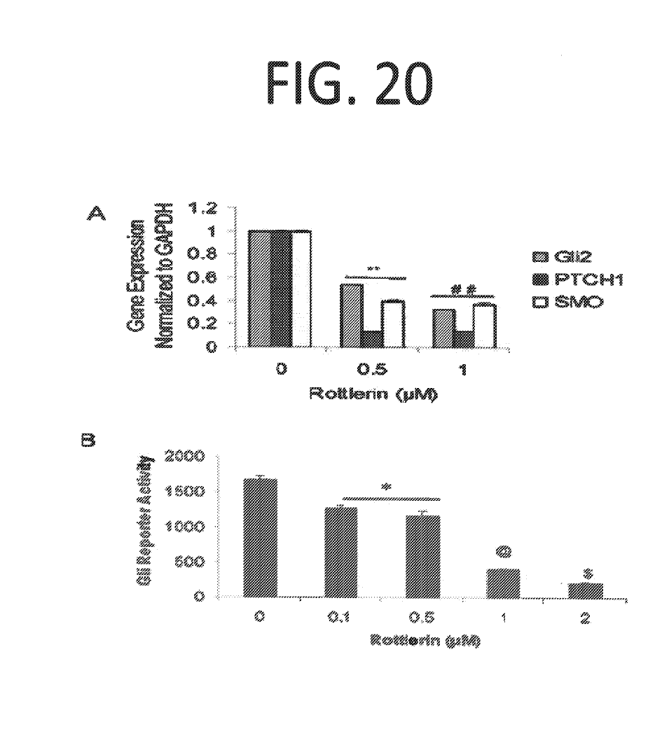

The Hedgehog (Hh) signaling pathway may be essential to the development of tissues and organs. Aberrant activation of sonic hedgehog (Shh) signaling pathway may play important roles in tumorigenesis and progression of several tumors. Therefore, the effects of rottlerin on the expression of Shh receptors (Patched-1, Smoothened) and effectors (Gli2) by qRT-PCR were examined. Rottlerin inhibited the expression of Patched-1, Smo and Gli2, as shown in FIG. 20A. Since Gli transcription factor may mediate the effects of Shh which may play important roles in maintaining stemness and tumorigenesis, the Gli transcriptional activity was measured, as shown in FIG. 20B. Rottlerin inhibited Gli transcriptional activity in a dose-dependent manner. The data suggests that rottlerin can regulate pancreatic carcinogenesis by inhibiting several signaling molecules of Shh pathway. Ptch 1 is the downstream target of Gli transcription factor.

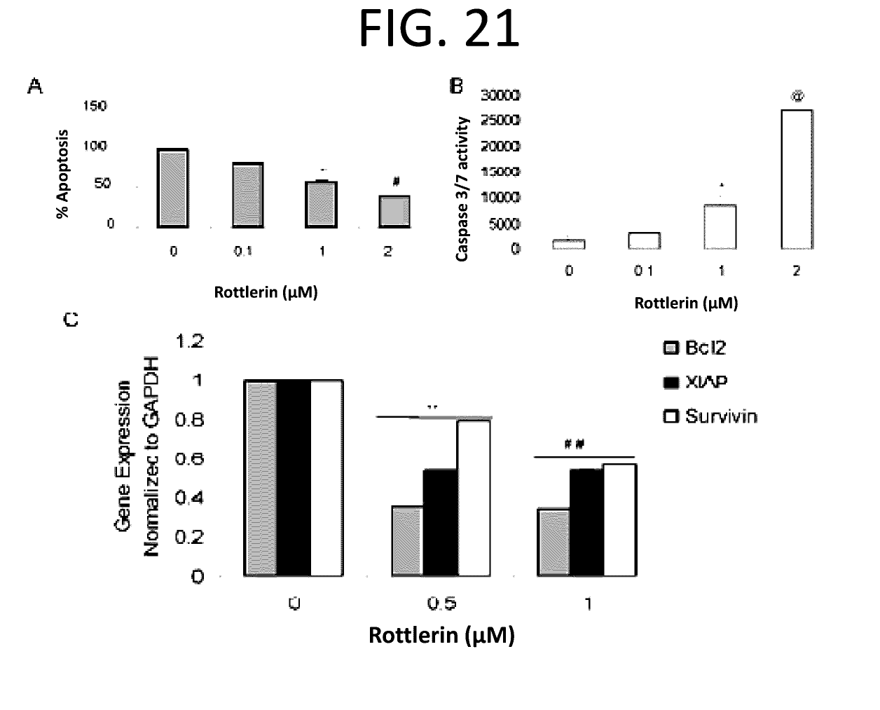

Rottlerin may activate caspase-3/-7, induce apoptosis, and inhibit the expression of Bcl-2, XIAP and Survivin in pancreatic CSCs. The effects of rottlerin on caspase-3/-7 activity, apoptosis, and expression of apoptosis related genes were examined, as shown in FIG. 21. Rottlerin induced apoptosis and caspase-3/-7 activity, as shown in FIGS. 21A and 21B. Since IAPs, Bcl-2 family members may play major roles in regulation of apoptosis, the effects of rottlerin on the expression of Bcl-2, XIAP, and Survivin were studied. Rottlerin inhibited the expression of Bcl-2, XIAP, and Survivin, as shown in FIG. 21C. The data suggests that rottlerin induces apoptosis in pancreatic CSCs through inhibition of apoptosis-related genes (Bcl-2, XIAP and Survivin), and induction of caspase-3/-7 activation respectively.

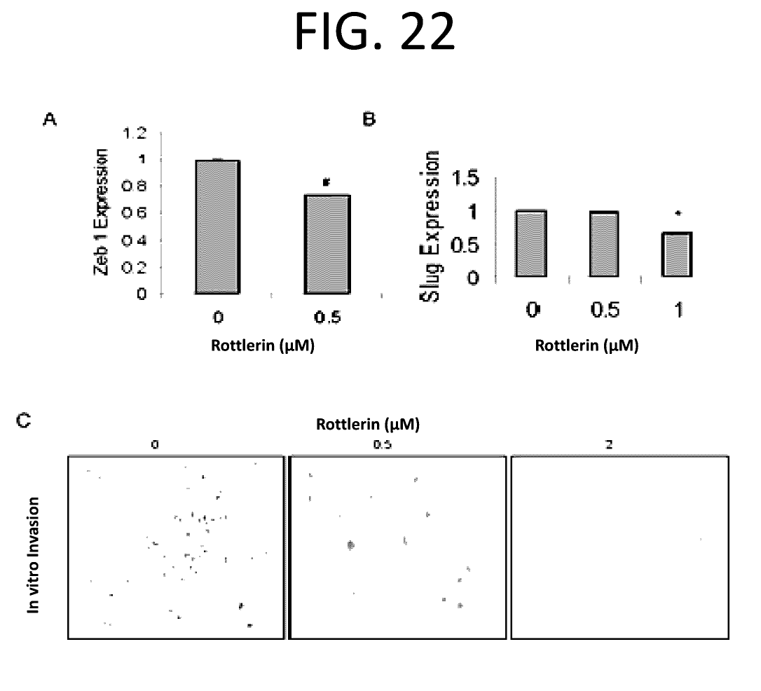

Rottlerin may inhibit epithelial-mesenchymal transition markers (EMT) and cancer stem cell viability in spheroids, invasion in human pancreatic CSCs. EMT may play a crucial role in tumorigenesis and cancer progression. Recent studies revealed that there may be a direct link between the EMT program and the gain of epithelial stem cell properties. EMT may be sufficient to induce a population with stem cell characteristics from well-differentiated epithelial cells and cancer cells. The effects of rottlerin on the expression of EMT transcription factors in pancreatic CSCs were examined, as shown in FIG. 22. Zeb-1 and Slug have been shown to be upregulated during EMT. Rottlerin inhibited the expression of Zeb-1 and Slug, as shown in FIGS. 22A and 22B. The data suggests that rottlerin can regulate EMT by inhibiting the expression of Zeb-1 and Slug in CSCs.

The effects of rottlerin on invasion were studied. Rottlerin inhibited the in vitro invasion of pancreatic CSCs, as shown in FIG. 22C. The data suggests that rottlerin can inhibit or reverse EMT by inhibiting ZEB1 and Slug.

### Example 4

The effects of stem cell inhibitors on brain cancer stem cells, prostate cancer stem cells, pancreatic cancer stem cells, and breast cancer stem cells were studied.

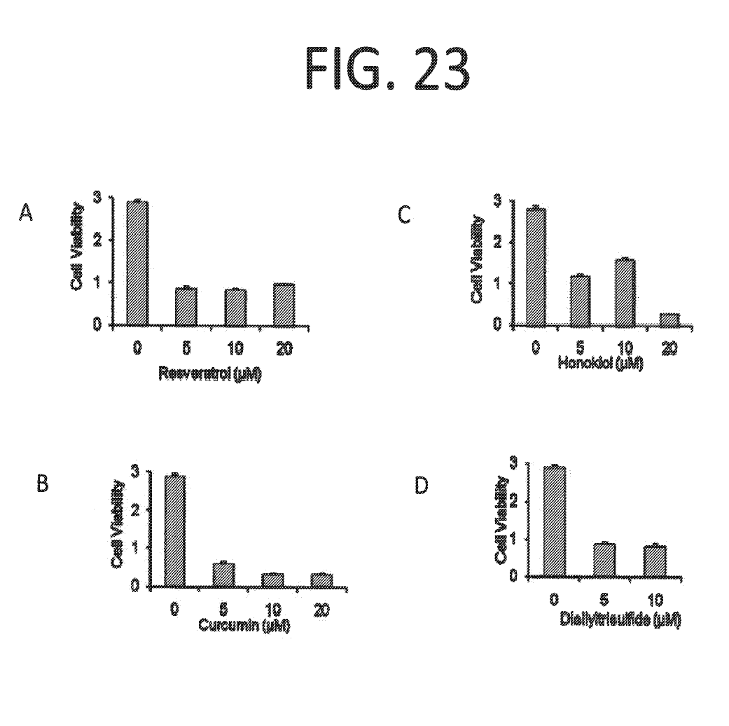

Brain CSCs were treated with resveratrol (0-20 μM), curcumin (0-20 μM) honokiol (0-20 μM), and diallyl trisulphide (0-10 μM) for 3 days and cell viability was measured by staining with trypan blue using Vi-CELL analyzer. The results of those studies are illustrated in FIG. 23.

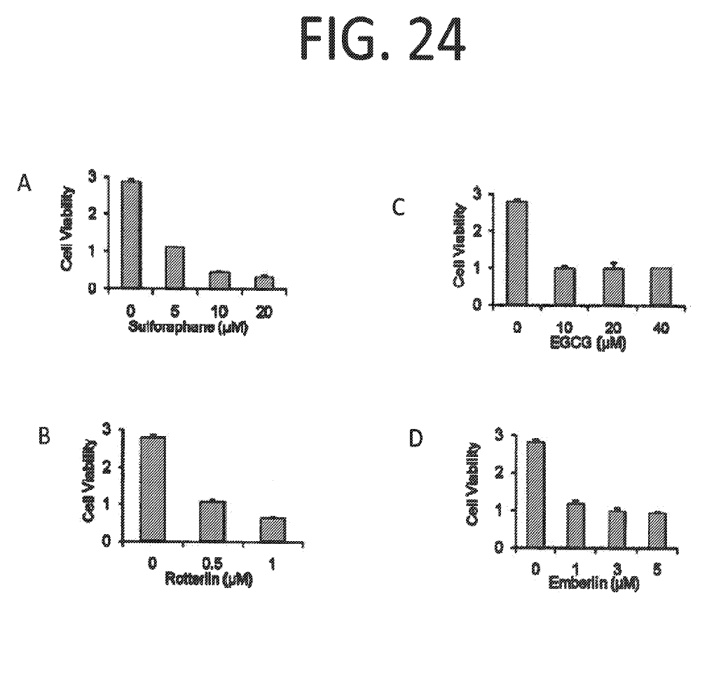

Brain CSCs were treated with sulforaphane (0-20 μM), rottlerin (0-1 μM), EGCG (0-40 μM), and embelin (0-5 μM) for 48 hours and cell viability was measured by staining with trypan blue using Vi-CELL analyzer. The results of those studies are illustrated in FIG. 24.

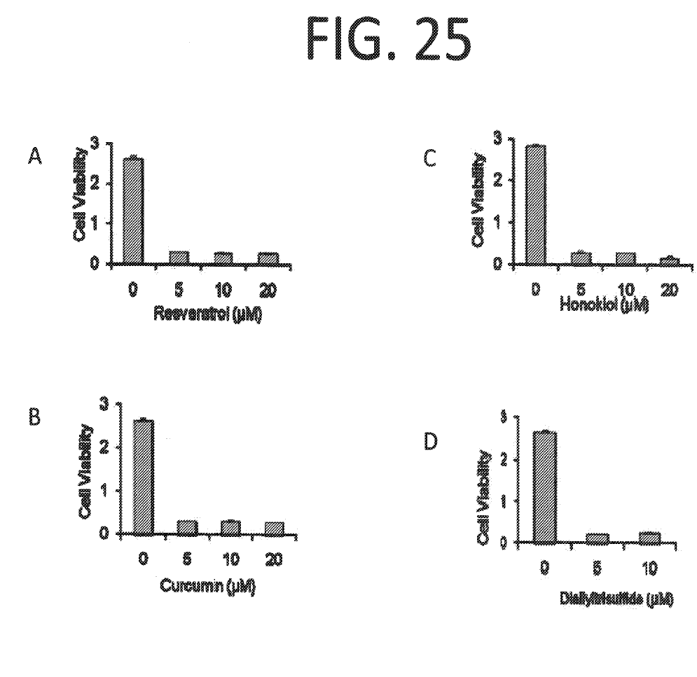

Prostate CSCs were treated with resveratrol (0-20 μM), curcumin (0-20 μM), honokiol (0-20 μM), and diallyl trisulphide (0-10 μM) for 3 days and cell viability was measured by staining with trypan blue using Vi-CELL analyzer. The results of those studies are illustrated in FIG. 25.

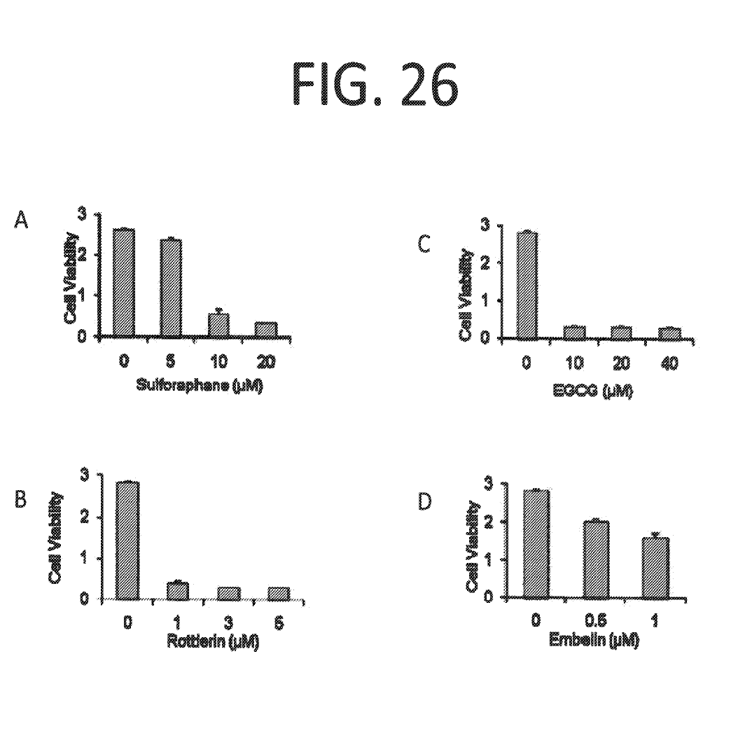

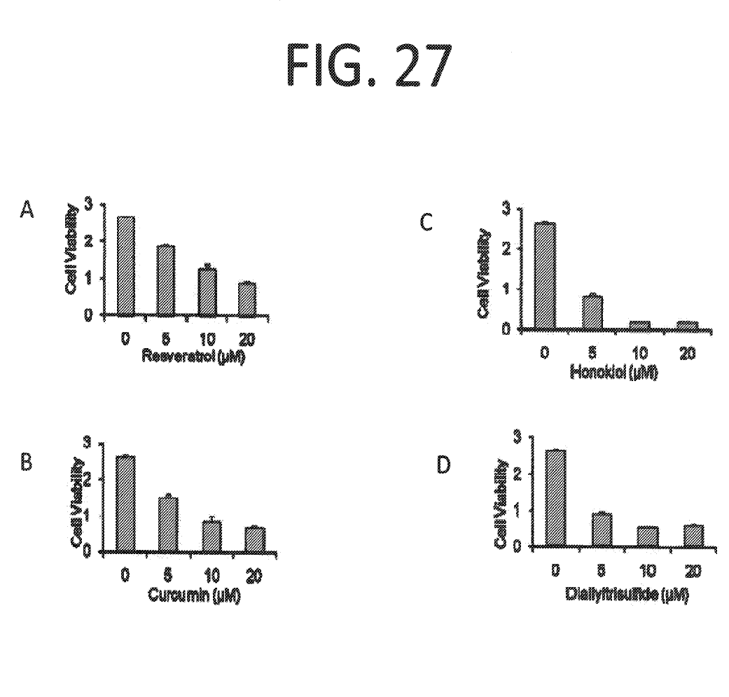

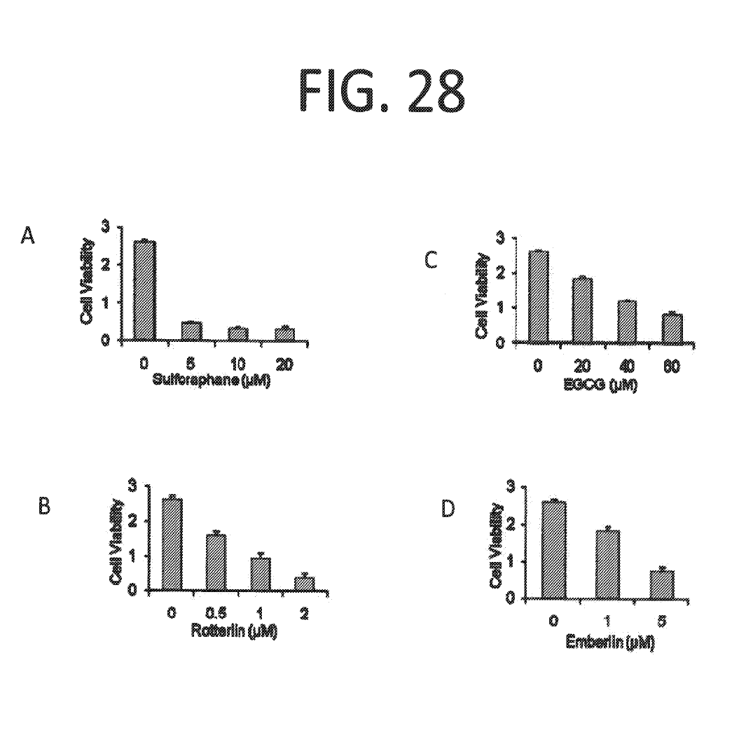

Prostate CSCs were treated with sulforaphane (0-20 μM), rottlerin (0-5 μM), EGCG (0-40 μM), and embelin (0-1 μM) for 3 days and cell viability was measured by staining with trypan blue using Vi-CELL analyzer. The results of those studies are illustrated in FIG. 26.

Pancreatic CSCs were treated with resveratrol (0-20 μM), curcumin (0-20 μM), honokiol (0-20 μM), and diallyl trisulphide (0-20 μM) for 3 days and cell viability was measured by staining with trypan blue using Vi-CELL analyzer. The results of those studies are illustrated in FIG. 27.

Pancreatic CSCs were treated with sulforaphane (0-20 μM), rottlerin (0-2 μM), EGCG (0-60 μM), and embelin (0-5 μM) for 3 days and cell viability was measured by staining with trypan blue using Vi-CELL analyzer. The results of those studies are illustrated in FIG. 28.

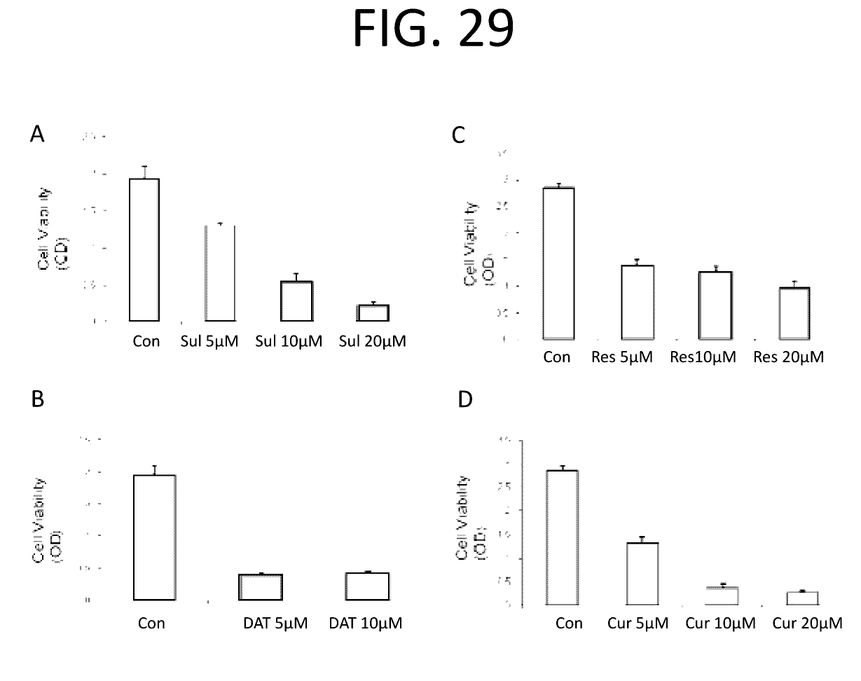

Breast CSCs were seeded in 96-well plate and treated with sulforaphane, diallyl trisulphide, resveratrol, and curcumin for 3 days and cell viability was measured by XTT assay. The results of those studies are illustrated in FIG. 29.

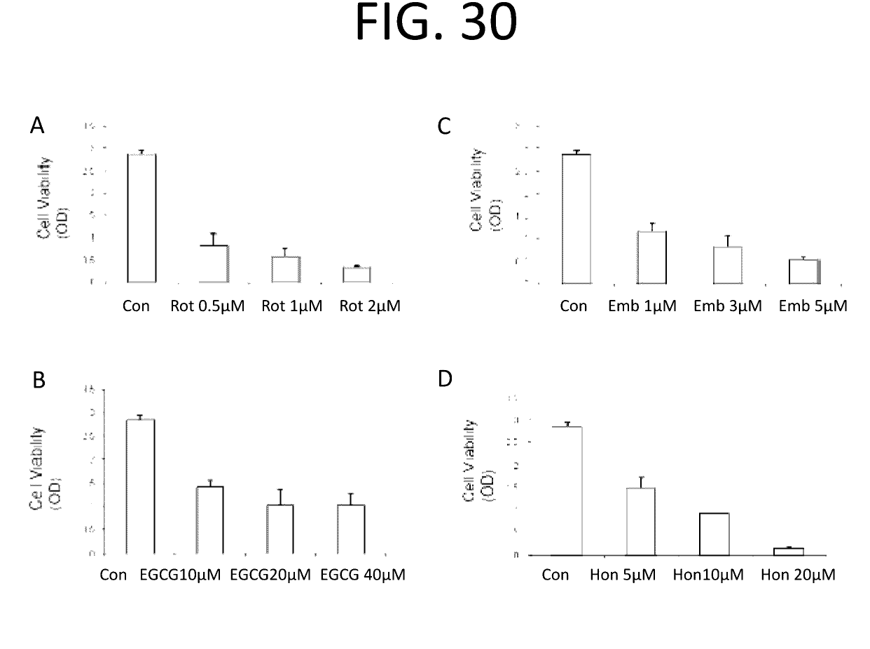

Breast CSCs were seeded in 96-well plate and treated with Rottlerin, EGCG, embelin, and honokiol for 3 days and cell viability was measured by XTT assay. The results of those studies are illustrated in FIG. 30.

### Example 5

The effects of chromatin modulators on pancreatic cancers stem cells were studied.

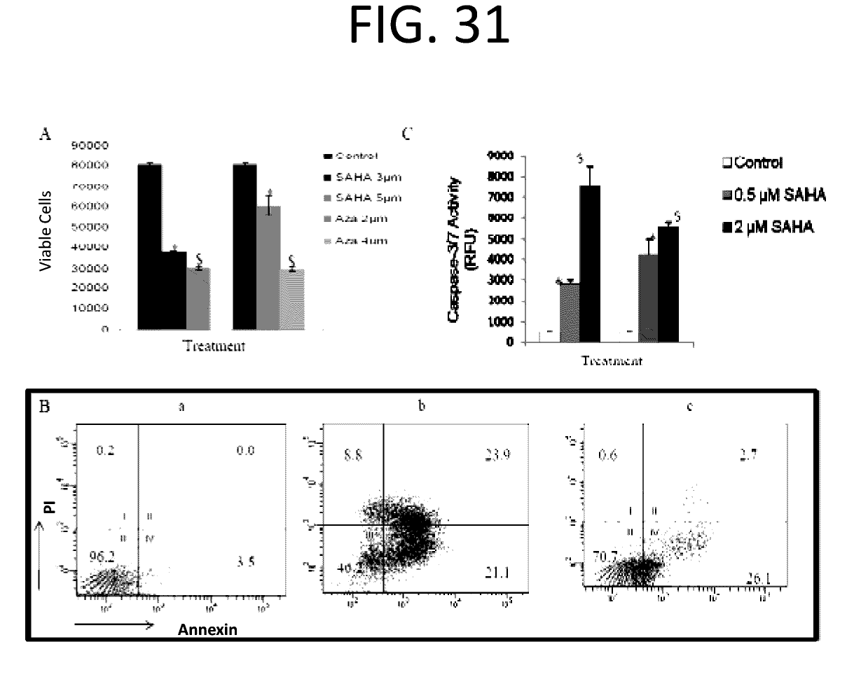

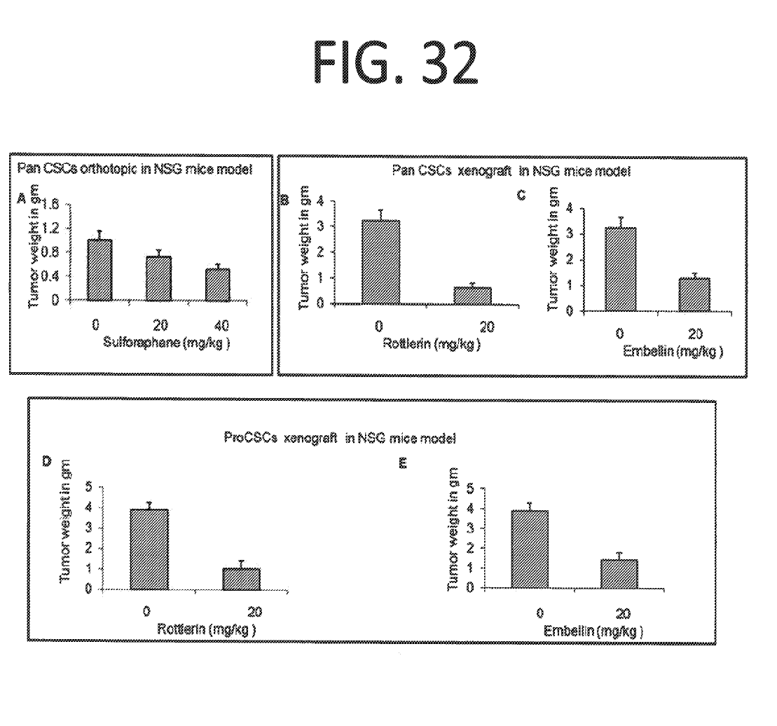

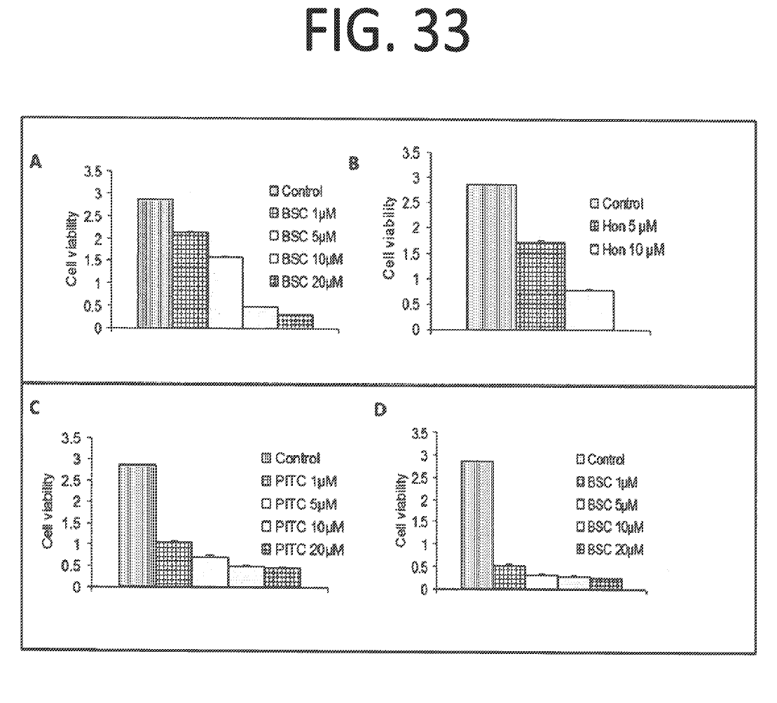

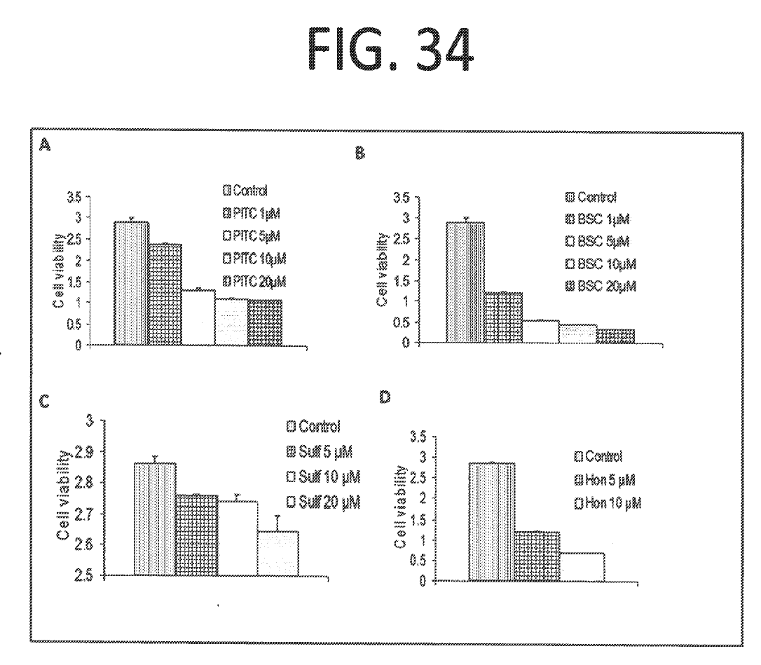

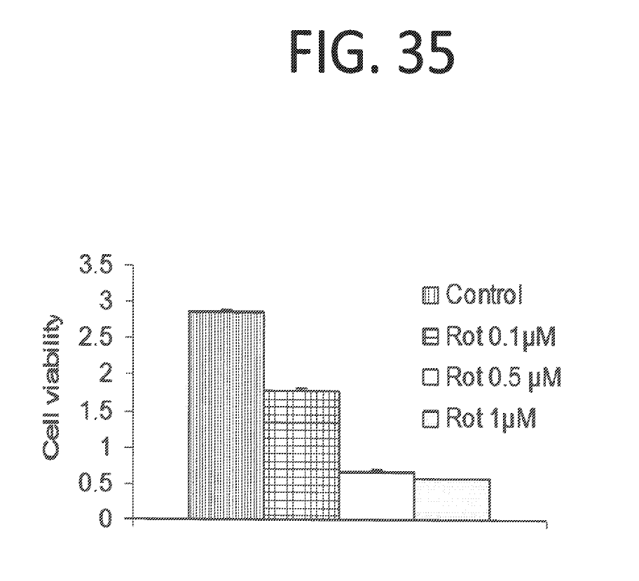

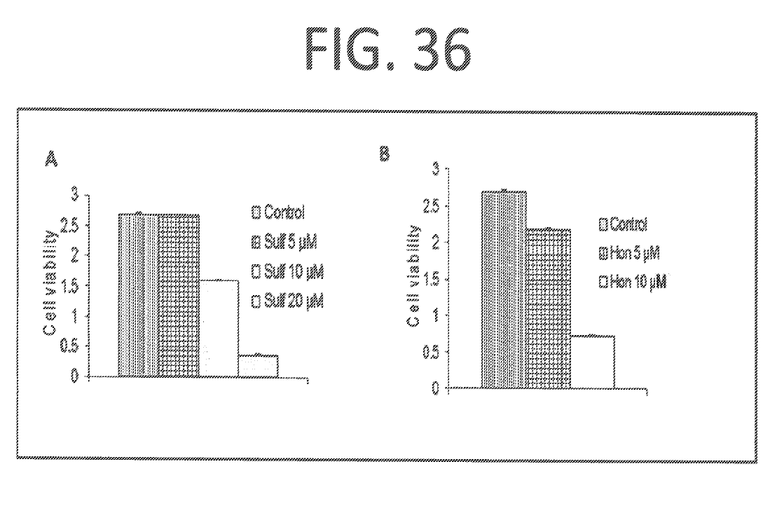

Pancreatic CSCs were treated with SAHA and Vorinostat (3 and 5 μM) and 5-Aza-2′-deoxycytidine (5-Aza-dC, 2 and 4 μM) and cell viability was measured at 48 hours by staining with trypan blue using Vi-CELL analyzer. The results of those studies are illustrated in FIG. 31A.

Pancreatic CSCs were (a) untreated, (b) treated with SAHA, or (c) treated with 5-Aza-dC for 48 hours and apoptosis was measured by staining with annexin-PI using Accuri Flow Cytometer. The results of those studies are illustrated in FIG. 31B.

Pancreatic CSCs were treated with SAHA (0.5 and 2 μM) or 5-Aza-dC (1 and 3 μM) for 24 hours and caspase-3/7 activity was measured. The results of those studies are illustrated in FIG. 31C.

### Example 6

### EGCG Inhibits the Formation of Primary and Secondary Tumor Spheroids and Colonies by Pancreatic Cancer Stem Cells

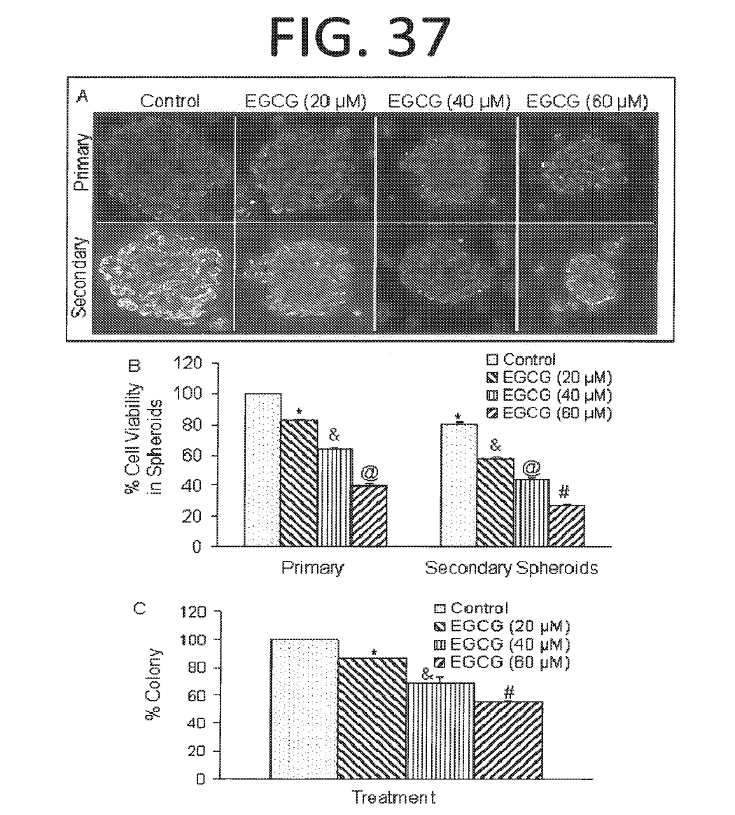

The ability of cells to self-renew is one of the main characteristics of CSCs. Therefore, it was examined whether EGCG inhibits the growth of CSCs isolated from human primary pancreatic tumors by measuring sphere formation and cell viability in those spheroids. CSCs were grown in pancreatic cancer stem cell defined medium in suspension, and treated with EGCG. At the end of incubation period, primary and secondary spheroids in each well were photographed. EGCG inhibited the growth (size) of spheroids in suspension in a dose dependent manner (FIG. 37A). The spheroids from each treatment group were collected and resuspended for counting cell viability. EGCG inhibited CSC's viability in primary and secondary spheroids in a dose-dependent manner (FIG. 37B). These data suggest that EGCG can be effective in inhibiting the growth of pancreatic CSCs.

Since EGCG inhibited the growth of tumor spheroid and cell viability of CSCs, the effects of EGCG on colony formation were examined (FIG. 37C). Pancreatic CSCs were grown in agar, and treated with various doses of EGCG for 3 weeks. At the end of incubation period, numbers of colonies were counted. EGCG inhibited the growth of colonies in a dose-dependent manner. These data suggest that EGCG can be effective in inhibiting the self-renewal capacity of pancreatic CSCs.

### EGCG Induces Caspase-3/7 Activity and Apoptosis, and Inhibits the Expression of Bcl-2, Survivin and XIAP in Human Pancreatic CSCs.

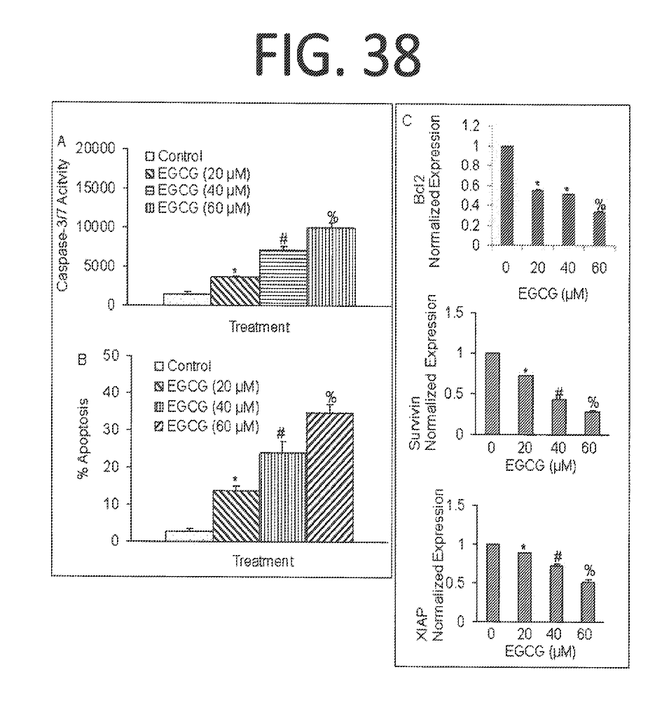

Since members of the IAP and Bcl-2 play important roles in cell survival and apoptosis (Srivastava R K. TRAIL/Apo-2L: mechanisms and clinical applications in cancer. Neoplasia 2001; 3:535-46), the effects of EGCG on caspase-3/7 activity and apoptosis, and on the expression of Bcl-2, survivin and XIAP in pancreatic CSCs were examined (FIG. 38). EGCG induced caspase-3/7 activity and apoptosis in pancreatic CSCs in a dose-dependent manner (FIGS. 38A and B). Furthermore, EGCG inhibited the expression of Bcl-2, survivin and XIAP in pancreatic CSCs (FIG. 38C). These data suggest that EGCG can induce apoptosis in CSCs by engaging cell-intrinsic pathway of apoptosis.

### EGCG Inhibits the Expression of Pluripotency Maintaining Transcription Factors, and Inhibition of Nanog Enhances the Inhibitory Effects of EGCG on Pancreatic CSC's Self-Renewal.

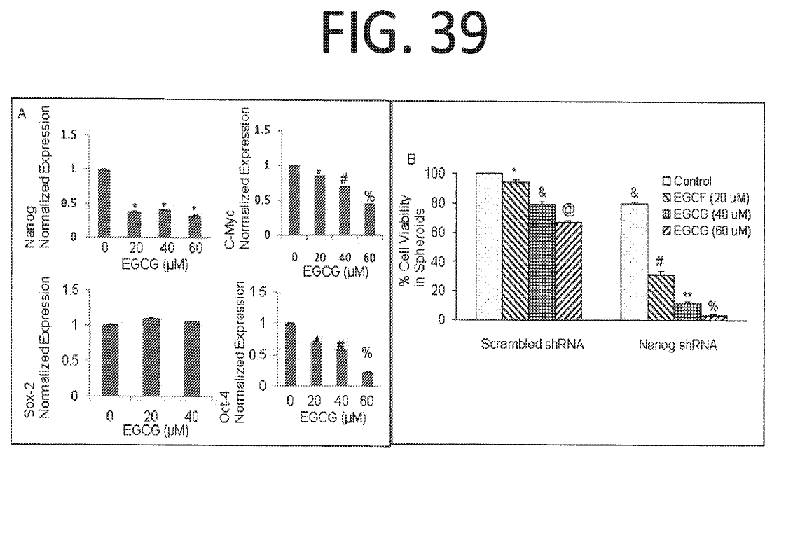

Since Nanog, Sox-2, c-Myc and Oct-4 are required for maintaining pluripotency in stem cells (Cavaleri F, Scholer H R. Nanog: a new recruit to the embryonic stem cell orchestra. Cell 2003; 113:551-2; Kashyap V, Rezende N C, Scotland K B, Shaffer S M, Persson J L, Gudas L J, Mongan N P. Regulation of stem cell pluripotency and differentiation involves a mutual regulatory circuit of the NANOG, OCT4, and SOX2 pluripotency transcription factors with polycomb repressive complexes and stem cell microRNAs. Stem Cells Dev 2009; 18:1093-108), the effects of EGCG on the expression of these factors were examined. As shown in FIG. 39A, EGCG inhibited the expression of Nanog, c-Myc and Oct-4 in pancreatic CSCs. However, EGCG has no effect on the expression of Sox-2.

A high level of Nanog is a key regulator of embryonic stem cell (ESC) self-renewal and puripotency. Jeter C R, Badeaux M, Choy G, Chandra D, Patrawala L, Liu C, Calhoun-Davis T, Zaehres H, Daley G Q, Tang D G. Functional evidence that the self-renewal gene NANOG regulates human tumor development. Stem Cells 2009; 27:993-1005. Nanog-deficient ES cells and embryos lose their pluripotency. Mitsui K, Tokuzawa Y, Itoh H, Segawa K, Murakami M, Takahashi K, Maruyama M, Maeda M, Yamanaka S. The homeoprotein Nanog is required for maintenance of pluripotency in mouse epiblast and ES cells. Cell 2003; 113:631-42. Since Nanog is highly expressed in CSCs compared to normal cells (Bae K M, Su Z, Frye C, McClellan S, Allan R W, Andrejewski J T, Kelley V, Jorgensen M, Steindler D A, Vieweg J, Siemann D W. Expression of pluripotent stem cell reprogramming factors by prostate tumor initiating cells. J Urol 2010; 183:2045-53), it was examined whether inhibition of Nanog by shRNA can enhance the inhibitory effects of EGCG on cell viability in spheroids. Lentiviral mediated transduction of Nanog shRNA inhibited Nanog protein expression (data not shown). EGCG inhibited CSC's viability in spheroids transduced with Nanog-scrambled shRNA in a dose-dependent manner (FIG. 39B). The inhibition of Nanog by shRNA further enhanced the antiproliferative effects of EGCG on CSCs. These data suggest that inhibition of Nanog may be an attractive target for regulation of self-renewal capacity of CSCs, and EGCG inhibits the factors required for maintaining pluripotency in CSCs.

### EGCG Inhibits Shh Signaling Pathway.

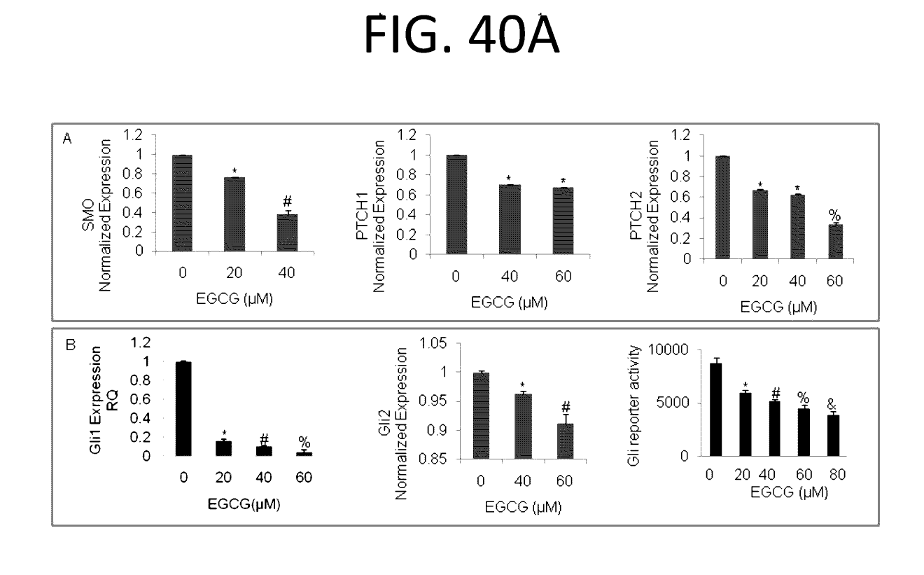

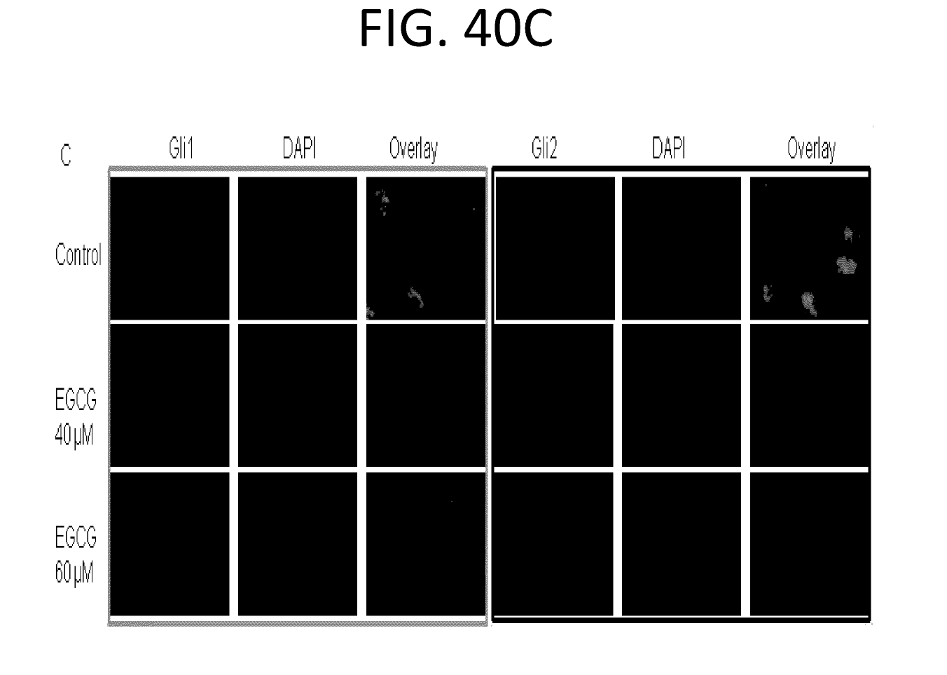

The effects of EGCG on the Shh pathway were examined by measuring the expression of Shh receptors (Patched-1, Patched-2 and Smoothened) and effectors (Gli1 and Gli2) by qRT-PCR (FIG. 40). EGCG inhibited the expression of smoothened (SMO), Patched-1, and Patched-2 (FIG. 40A). Similarly, EGCG inhibited the expression of transcription factor Gli1 and Gli2 (FIG. 40B). Since Gli mediates the effects of Shh which play important roles in maintaining stemness and tumorigenesis (Varjosalo M, Taipale J. Hedgehog: functions and mechanisms. Genes Dev 2008; 22:2454-72), the Gli transcriptional activity was measured by luciferase assay. As shown in FIG. 40B, EGCG inhibited Gli transcriptional activity in a dose-dependent manner. EGCG inhibited the expression of Ptch 1 and Ptch 2 because they are downstream targets of Gli.

The effects of EGCG on nuclear expression of Gli1 and Gli2 were next examined by immunohistochemistry (FIG. 40C). EGCG inhibited the nuclear expression of Gli1 and Gli2 proteins. These data suggest that EGCG can inhibit pancreatic CSC characteristics by inhibiting Shh pathway which has been shown to play an important role in maintaining stemness and metastasis.

### EGCG Inhibits the Expression of Epithelial-Mesenchymal Transition (EMT) Markers, Migration, Invasion and TCF/LEF Activity.

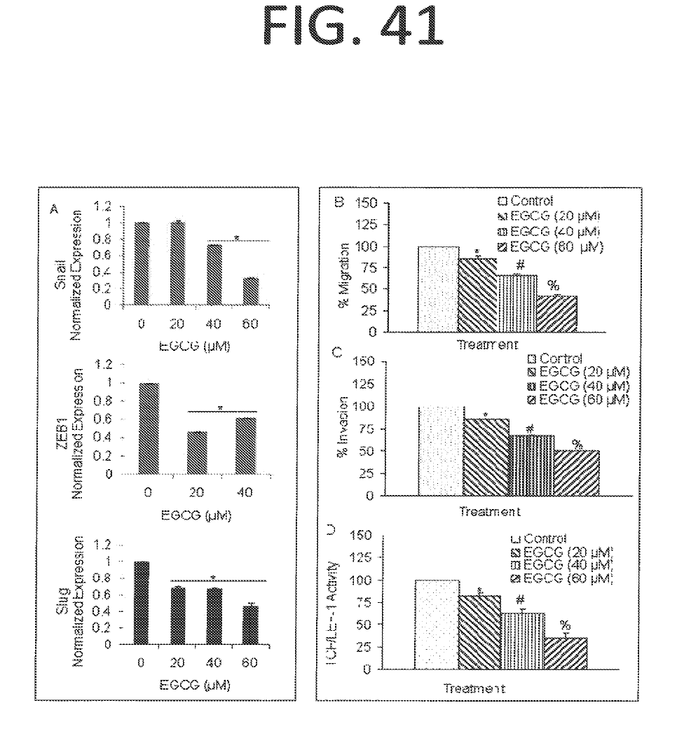

During cancer metastasis, the mobility and invasiveness of cancer cells increase. To detach from neighboring cells and invade adjacent cell layers, carcinoma cells must lose cell-cell adhesion and acquire motility. The highly conserved EMT program has been implicated in dissemination of carcinoma cells from primary epithelial tumors. Thiery J P, Acloque H, Huang R Y, Nieto M A. Epithelial-mesenchymal transitions in development and disease. Cell 2009; 139:871-90. Tumor progression is frequently associated with the downregulation of E-cadherin (Thiery J P, Acloque H, Huang R Y, Nieto M A. Epithelial-mesenchymal transitions in development and disease. Cell 2009; 139:871-90), and upregulation of vimentin and several transcription factors including Snail, ZEB1 and Slug. Iwatsuki M, Mimori K, Yokobori T, Ishi H, Beppu T, Nakamori S, Baba H, Mori M. Epithelial-mesenchymal transition in cancer development and its clinical significance. Cancer Sci 2010; 101:293-9. Cancer stem cells undergoing metastasis usually express EMT markers. The regulation of EMT markers by EGCG was therefore examined. As expected, EGCG inhibited the expression of Snail, ZEB 1 and Slug as measured by q-RT-PCR (FIG. 41A).

Since CSCs appear to play a significant role in early metastasis (Mueller M T, Hermann P C, Heeschen C. Cancer stem cells as new therapeutic target to prevent tumour progression and metastasis. Front Biosci (Elite Ed) 2010; 2:602-13), the effects of EGCG on migration and invasion of CSCs were measured (FIGS. 40B and 40C). EGCG inhibited cell migration and invasion of CSCs. These data suggest that EGCG can inhibit early metastasis of pancreatic CSCs.

Wnt/β-catenin signaling involves target gene activation by a complex of β-catenin with a T-cell factor (TCF) family member. Increased expression of β-catenin has been associated with enhanced transcriptional activation of TCF/LEF, invasion and migration by CSCs. The effects of EGCG on TCF/LEF transcriptional activity were therefore examined by luciferase assay (FIG. 41D). As expected, EGCG inhibited TCF/LEF activity in pancreatic CSCs. These data suggest that inhibition of EMT markers by EGCG could inhibit early metastasis of CSCs.

### Quercetin Enhances the Effects of EGCG on Spheroid and Colony Formation, Apoptosis, Invasion, Migration, and the Transcriptional Activities of TCF/LEF and Gli in Pancreatic CSCs.

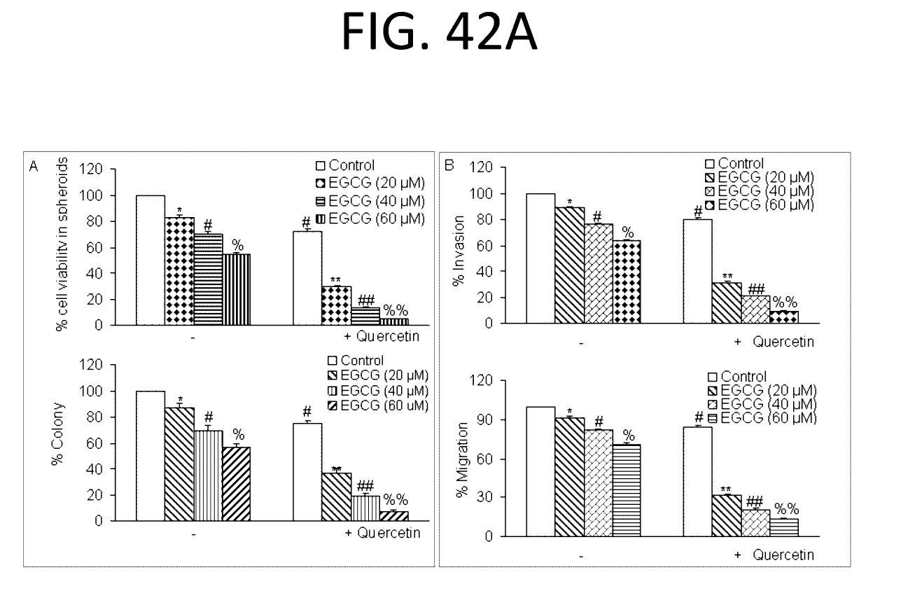

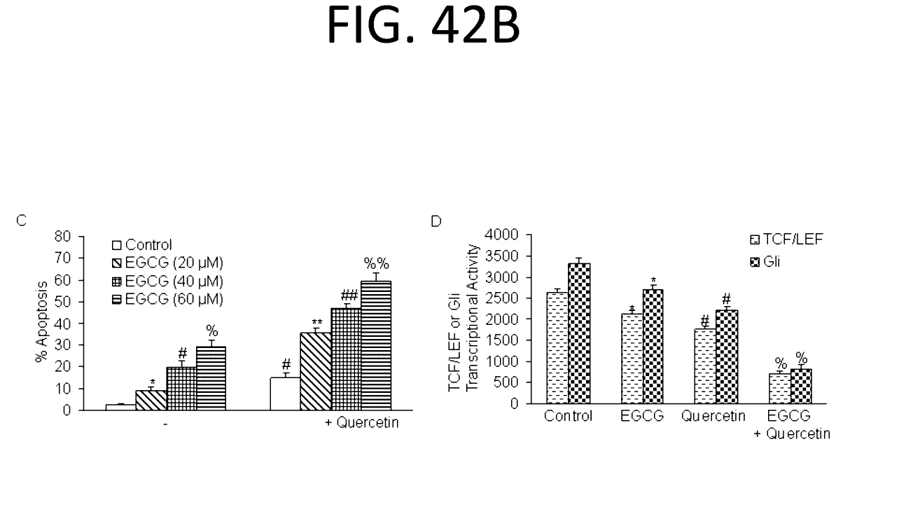

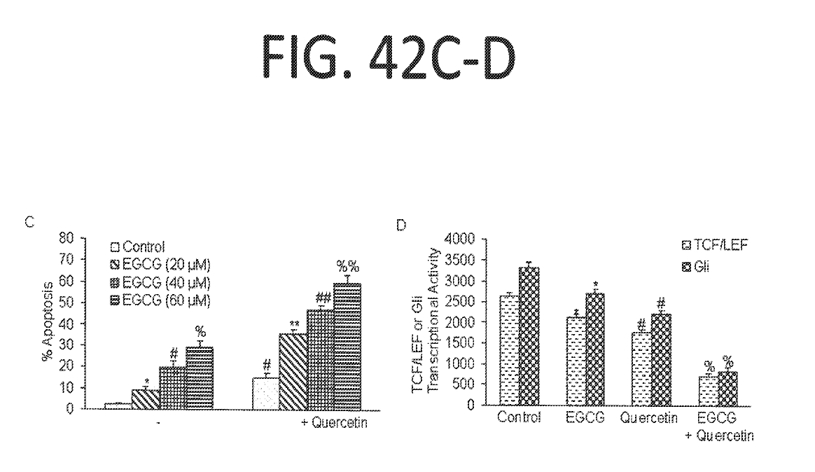

That quercetin can enhance the inhibitory effects of sulforaphane on CSC's characteristics was recently demonstrated. Srivastava R K, Tang S N, Zhu W, Meeker D, Shankar S. Sulforaphane synergizes with quercetin to inhibit self-renewal capacity of pancreatic cancer stem cells. Front Biosci (Elite Ed) 2011; 3:515-28; Tang S N, Singh C, Nall D, Meeker D, Shankar S, Srivastava R K. The dietary bioflavonoid quercetin synergizes with epigallocathechin gallate (EGCG) to inhibit prostate cancer stem cell characteristics, invasion, migration and epithelial-mesenchymal transition. J Mol Signal 2010; 5:14. It was therefore examined whether quercetin enhances the inhibitory effects of EGCG on self-renewal, migration and invasion of pancreatic CSCs (FIG. 42). EGCG inhibited cell viability in spheroids, colony formation, migration and invasion by CSCs in a dose-dependent manner (FIGS. 42A and 42B). Quercetin, although effective alone, further enhanced the inhibitory effects of EGCG on cell viability, colony formation, migration and invasion. Furthermore, EGCG and quercetin alone induced apoptosis (FIG. 42C). Interestingly, EGCG synergizes with quercetin to induce apoptosis in pancreatic CSCs. These data suggest that EGCG can be used with quercetin to inhibit pancreatic CSC characteristics.

Since enhanced levels of TCF/LEF and Gli transcriptional activities have been associated with CSC characteristics, the expression of TCF/LEF and Gli activities in pancreatic CSCs was measured (FIG. 42D). EGCG inhibited both TCF/LEF and Gli transcriptional activities in pancreatic CSCs. These data suggest that EGCG synergizes with quercetin to inhibit self-renewal capacity of pancreatic CSCs by inhibiting TCF/LEF and Gli transcription factors.

### Example 7

### Quercetin Enhances the Effects of Sulforaphane on Spheroid and Colony Formation by Pancreatic Cancer Stem Cells

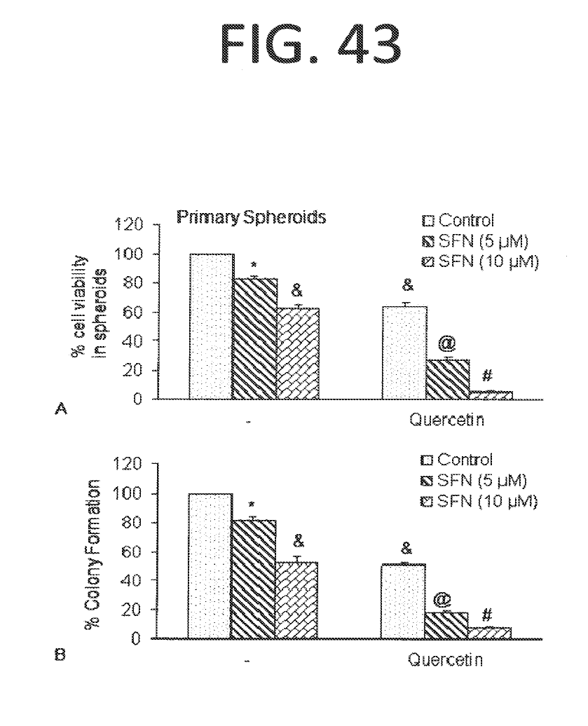

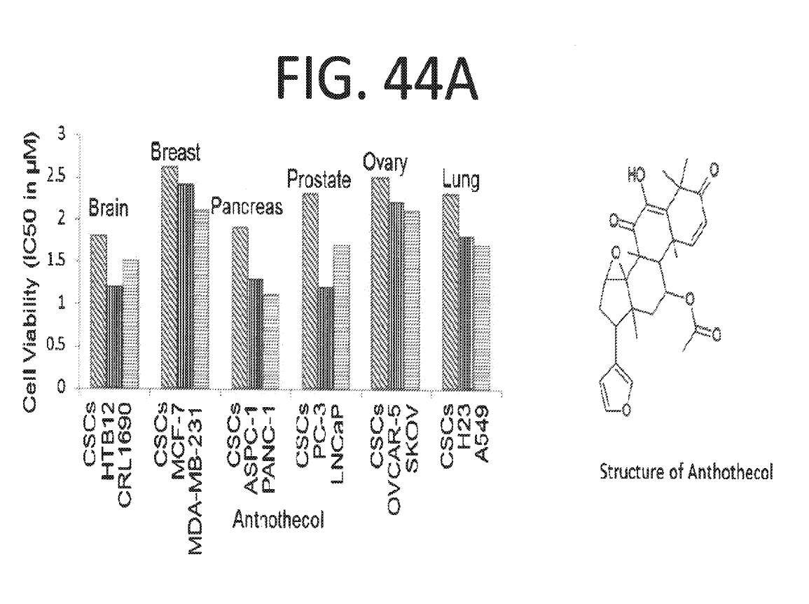

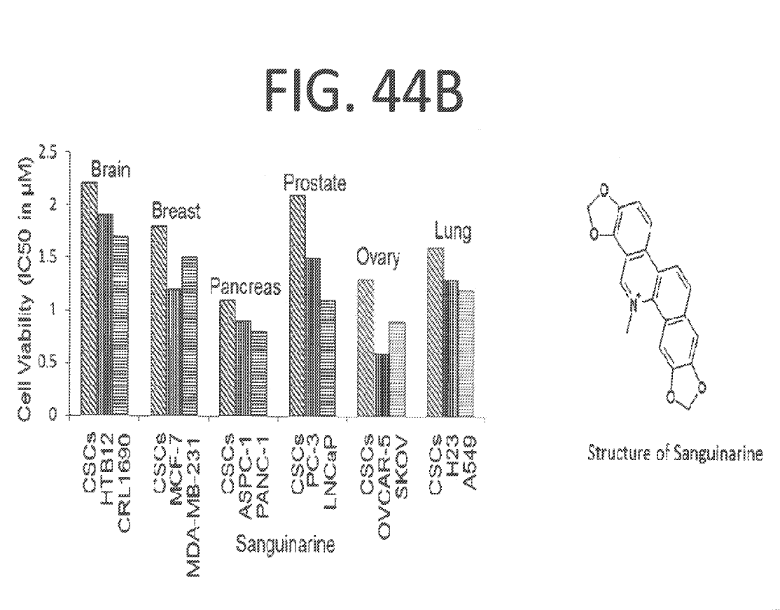

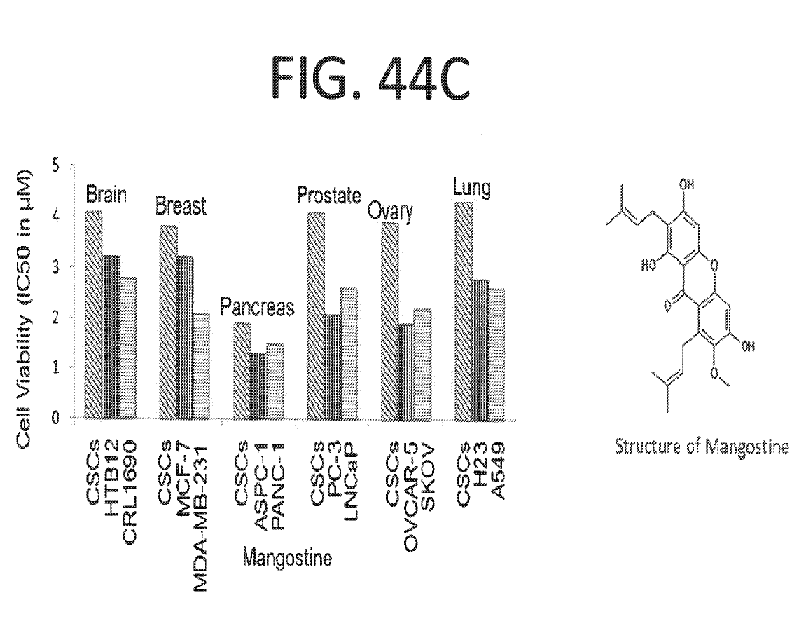

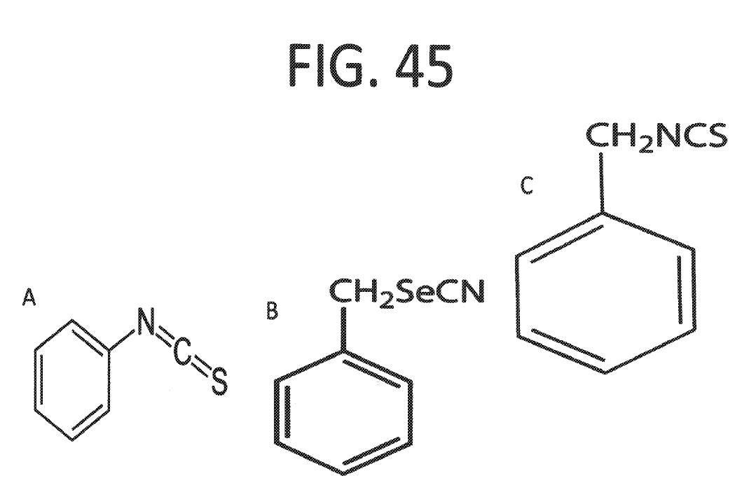

Quercetin has been shown to enhance the effects of anticancer drugs and sensitize cancer cells to chemotherapy and radiotherapy. It was therefore examined whether quercetin enhances the effects of sulforaphane (SFN) on spheroid and colony formation by pancreatic CSCs (FIG. 43). SFN inhibited the cell viability and colony formation of pancreatic CSCs in a dose-dependent manner. Quercetin, although effective alone, further enhanced the biological effects of SFN on cell viability (in spheroids) and colony formation. These data suggest that quercetin can be used with SFN to selectively target pancreatic CSCs.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

All references, including publications, patent applications, and patents, cited herein are hereby incorporated by reference to the same extent as if each reference was individually and specifically indicated to be incorporated by reference and was set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating or preventing a cancer by targeting and inhibiting cancer stem cells, comprising administering to a subject in need thereof a pharmaceutically effective amount of a pharmaceutical composition and a vehicle for inhibiting the cancer stem cells.

2. The method of treating or preventing a cancer by targeting and inhibiting cancer stem cells as claimed in claim 1, wherein the pharmaceutical composition comprises sulforaphane, resveratrol, mangostine, honokiol and diallyltrisulphide.

3. The method of treating or preventing a cancer by targeting and inhibiting cancer stem cells as claimed in claim 1, wherein the pharmaceutical composition comprises sulphoraphane, resveratrol and gemcitabine.

4. The method of treating or preventing a cancer by targeting and inhibiting cancer stem cells as claimed in claim 1, wherein the pharmaceutical composition comprises mangostine, resveratrol and gemcitabine.

5. The method of treating or preventing a cancer by targeting and inhibiting cancer stem cells as claimed in claim 1, wherein the pharmaceutical composition comprises honokiol, resveratrol and gemcitabine.

6. The method of treating or preventing a cancer by targeting and inhibiting cancer stem cells as claimed in claim 1, wherein the vehicle is selected from the group consisting of liposomes, micelles, polymers, dendrimers, biodegradable particles, artificial DNA nanostructure, carbohydrate-based nanoparticles, lipid-based nanoparticles, carbon nanoparticles and gold nanoparticles.

7. The method of treating or preventing a cancer by targeting and inhibiting cancer stem cells as claimed in claim 1, wherein the vehicle comprises a targeting agent and an imaging agent.

8. The method of treating or preventing a cancer by targeting and inhibiting cancer stem cells as claimed in claim 1, wherein the vehicle is carbon nanoparticles or gold nanoparticles.

9. The method of treating or preventing a cancer by targeting and inhibiting cancer stem cells as claimed in claim 2, wherein sulforaphane is from 0.1 mg/kg of body weight/day to 300 mg/kg of body weight/day, resveratrol is from 0.1 mg/kg of body weight/day to 300 mg/kg of body weight/day, mangostine is from 0.1 mg/kg of body weight/day to 300 mg/kg of body weight/day, honokiol is from 0.1 mg/kg of body weight/day to 300 mg/kg of body weight/day and diallyltrisulphide is from 0.1 mg/kg of body weight/day to 300 mg/kg of body weight/day.

10. The method of treating or preventing a cancer by targeting and inhibiting cancer stem cells as claimed in claim 3, wherein sulphoraphane is from 0.1 mg/kg of body weight/day to 500 mg/kg of body weight/day, resveratrol is from 0.1 mg/kg of body weight/day to 500 mg/kg of body weight/day and gemcitabine is from 1.0 μg/kg of body weight/day to 100 mg/kg of body weight/day.

11. The method of treating or preventing a cancer by targeting and inhibiting cancer stem cells as claimed in claim 4, wherein mangostine is from 0.1 mg/kg of body weight/day to 500 mg/kg of body weight/day, resveratrol is from 0.1 mg/kg of body weight/day to 500 mg/kg of body weight/day and gemcitabine is from 1.0 μg/kg of body weight/day to 100 mg/kg of body weight/day.

12. The method of treating or preventing a cancer by targeting and inhibiting cancer stem cells as claimed in claim 5, wherein honokiol is from 0.1 mg/kg of body weight/day to 500 mg/kg of body weight/day, resveratrol is from 0.1 mg/kg of body weight/day to 500 mg/kg of body weight/day and gemcitabine is from 1.0 μg/kg of body weight/day to 100 mg/kg of body weight/day.

13. The method of treating or preventing a cancer by targeting and inhibiting cancer stem cells as claimed in claim 9, wherein a molar ratio of the sulforaphane, resveratrol, mangostine, honokiol, and diallyltrisulphide is 1 to 30%, 1 to 30%, 1 to 30%, 1 to 30%, and 1 to 30%, respectively.

14. The method of treating or preventing a cancer by targeting and inhibiting cancer stem cells as claimed in claim 10, wherein a molar ratio of the sulforaphane, resveratrol, and gemcitabine is 1 to 45%, 1 to 45%, and 0.1 to 30%, respectively.

15. The method of treating or preventing a cancer by targeting and inhibiting cancer stem cells as claimed in claim 11, wherein a molar ratio of the mangostine resveratrol, and gemcitabine will be about 1 to 45%, 1 to 45%, and 0.1 to 30%, respectively.

16. The method of treating or preventing a cancer by targeting and inhibiting cancer stem cells as claimed in claim 12, wherein a molar ratio of the honokiol, resveratrol, and gemcitabine will be about 1 to 45%, 1 to 45%, and 0.1 to 30%, respectively.
